(19) United States
(12) Patent Application Publication
(10) Pub. No.: US 2013/0244948 A1
(43) Pub. Date: Sep. 19, 2013

(54) COMPOSITIONS OF CELLS, MEDIA, AND METHODS THEREOF

(71) Applicant: David W. Scharp, Irvine, CA (US)

(72) Inventor: David W. Scharp, Irvine, CA (US)

(73) Assignee: Scharp Technologies, Inc., Irvine, CA (US)

(21) Appl. No.: 13/843,034

(22) Filed: Mar. 15, 2013

Related U.S. Application Data
(60) Provisional application No. 61/611,508, filed on Mar. 15, 2012.

(51) Int. Cl. C12N 5/0775; A61K 38/02; A61K 35/54; A61K 35/28

(52) U.S. Cl. CPC C12N 5/0675; A61K 35/28; A61K 38/02; A61K 35/545
USPC 514/18.8; 435/347; 435/373; 514/18.6

(57) ABSTRACT

The disclosure provides a composition that comprises co-culture of small embryonic-like stem cells and mesenchymal stem cells, where cell media is reduced or lacking in exogenously supplied growth factors, as well as compositions of growth media that result from, or are manufactured by, co-culture of at least two types of different cells. Also provided are methods for selecting cell media that meet criteria pertaining to cell migration (gap assays) and to confluence.

### RELATED APPLICATIONS

The present application claims the full benefit Paris Convention benefit of and priority to U.S. Provisional Application Ser. No. 61/611,508 filed on Mar. 15, 2012, and expressly incorporates the contents by reference as if fully set forth herein.

### FIELD OF THE DISCLOSURE

The present disclosure relates to compositions of cells, to combinations of cells grown together in culture, to methods for enhancing cell yield, to cell compositions that are produced using culture media lacking in growth factors, and to the manufacture of enhanced culture media by growing cells in co-culture.

### BACKGROUND OF THE DISCLOSURE

Pharmaceutical treatments that administer precursor cells, or that administer small molecules that modulate the biology of precursor cells present in the body, are useful for treating numerous indications, comprising cardiovascular diseases, e.g., ischemic-damaged tissue, genetic diseases, cancer, diseases of the nervous system, e.g., spinal cord injury, and most other organ systems. These precursor cells occur in various categories, including embryonic stem cells, and tissue-specific lineage-committed progenitor cells.

Stem cells are one type of precursor cell. From the first stages of embryonic development to adulthood, the stem cells in the body are hierarchically organized according to the differentiation capacity of the cell. The most primitive stem cells are in the zygote (the fusion of two gametes that occurs in fertilization). The “totipotent” stem cells of the zygote have the ability to give rise to all the tissues of the embryo. At the morula stage of the embryo, cells have lost their totipotency, but have preserved their ability to differentiate into cells of all three embryonic germ layers (endoderm, ectoderm, mesoderm). Stem cells of these three layers are characterized as “pluripotent” stem cells. At later stages of embryonic development, germ layer-specific stem cells are called “multipotent,” where these multipotent stem cells can give rise to monopotent stem cells, that is, to stem cells that are committed to a specific type of tissue. While totipotent and pluripotent stem cells have been well characterized in the early embryonic stages, recent data establishes their presence in adult tissues, for example, in the form of cells called very small embryonic-like stem cells (VSELs) (see, e.g., Zuba-Surma et al (2009) Cytometry A. 75:4-13).

Tissue-specific lineage-committed progenitor cells include precursor cells for skeletal muscle, precursor cells for cartilage, precursor cells for bone, precursor cells for adipose tissue, precursor cells for the central nervous system, and precursor cells for the gut mucosa. Other categories of precursor cells exist, namely, lineage-uncommitted pluripotent stem cells (CD10+, CD13+, CD66e−, MHC I+), and lineage-uncommitted precursor cells (CD10+, CD13−, CD66e+, MHC I−) (see, e.g. Young and Black (2004) Anatomical Record Part A. 276A:75-102). Pluripotent stem cells can be further divided into mesodermal (mesenchymal) stem cells (MSC), epiblastic-like stem cells, ectodermal stem cells, neuroectodermal stem cells, and endodermal stem cells.

### Identifying Mesenchymal Stem Cells (MSCs)

In the present disclosure, the abbreviation “MSC” refers only to mesenchymal stem cells. Dominici et al (2006) Cytotherapy 8:315-317, discloses a definition for these cells, where the definition includes expressed markers, markers that must not be expressed (negative markers), and functions. The defining functions of MSC are adherency to a plastic surface and ability to differentiate to osteoblasts, adipocytes, and chondroblasts in vitro. According to Dominici et al, MSC must express CD105; CD73; and CD90, and must not express the following markers (negative markers). Negative markers are CD45, CD34, CD14 (macrophage marker), CD11b (macrophage marker), CD79alpha (B cell marker), CD19 (B cell marker), and HLA-DR. HLA-DR is not expressed on MSC unless stimulated, for example, by interferon-gamma. Only one of the two macrophage and B cell markers needs to be tested. To reiterate, the definition of Dominici et al for MSCs consists of requirements for plastic adhesion, positive and negative markers, and ability to differentiate.

DiGirolamo et al (1999) Brit. J. Haematol. 107:275-281, disclose that MSCs have been known by various different names, that is, plastic-adherent cells, colony-forming-units fibroblasts, mesenchymal stem cells, mesenchymal progenitor cells, and marrow stromal cells. The term “mesenchymal stem cell” refers to the ability of the cell to differentiate into cells of the mesenchymal cell lineages, while the term “mesenchymal stromal cell” refers to the cells belonging to the stroma that has a physical supporting role to the hematopoietic stem cell niche (Aguello et al (2010) Eur. Cell Mater. 20:121-133).

The mesoderm, which is one of the three embryonic germ layers (ectoderm; mesoderm; endoderm), can be a source of MSC cells (see, e.g., Vodyanik et al (2010) Cell Stem Cell. 7:718-729). MSCs can be used to treat disorders of the bone (Horwitz et al (1999) Nature Medicine 5:309-313), to treat spinal cord injury (Wright et al (2011) Stem Cells. 29:169-178); to reduce pathological immune responses, for example, in the treatment of organ transplants, or for the treatment of graft-versus-host disease (GVHD) (Berman et al (2010) Diabetes. 59:2558-2568). In some cases, the efficacy of MSCs can be due to a combination of anti-immune response effects and effects that actively encourage regeneration.

The bone marrow, in particular, has been used as a source of precursor cells, including hematopoietic stem/progenitor cells, mesenchymal stem cells, and endothelial progenitor cells, multipotent adult progenitor cells, and embryonic-like stem cells (VSELs) (see, e.g., Ratajczak et al (2011) Exp. Hematol. 39:225-237).

The present disclosure provides novel co-cultures of mesenchymal stem cells (MSCs) and very small embryonic-like stem cells (VSELs), and compositions that comprise expanded MSCs, or expanded VSELs.

### SUMMARY OF THE DISCLOSURE

Briefly stated, the disclosure provides a composition that comprises co-culture of small embryonic-like stem cells and mesenchymal stem cells, where cell media is reduced or lacking in exogenous growth factors, as well as compositions of growth media that result from, or are manufactured by, co-culture of at least two types of different cells. Also provided are methods for selecting cell media that meet criteria pertaining to cell migration (gap assays) and confluence.

The present invention provides a composition comprising cultured cells in a medium, further comprising a first population of cultured cells in the medium that is at least 80% mesenchymal stem cells (MSCs) and a second population of cells in the medium that is at least 80% VSELs. What is further provided is the above composition, wherein each of the MSCs are CD105+, CD90+, CD73+, CD44+. Also provides is the above composition, wherein each of the VSELs are SSEA-4+, Oct-4+, and Nanog+. Moreover, what is also provided is the above composition, comprising a first population of cultured cells in the medium that is at least 90% mesenchymal cells (MSCs) and a second population of cells in the medium that is at least 90% VSELs. In another aspect, what is provided is the above composition, wherein the medium is supplemented with fetal bovine serum.

What is also embraced is the above composition, wherein the MSCs form filopodia or processes that contact the VSELs, wherein the VSELs form filopodia or processes that contact the MSCs, or wherein the MSCs form filopodia or processes that contact the VSELs and the VSELs form filopodia or processes that contact the MSCs. In another aspect, what is provided is the above composition, comprising a plurality of cells, wherein the plurality of cells consists of a first population of cultured cells in the medium that is at least 80% mesenchymal cells (MSCs) and a second population of cells in the medium that is at least 80% VSELs. In yet another aspect, what is provided is the above composition, wherein the medium is not supplemented with any purified exogenous growth factor. What is also embraced is the above composition, wherein the medium is not supplemented with purified fibroblast growth factor (FGF), or with purified hepatocyte growth factor (HGF), or with any analogue thereof.

In composition embodiments, what is provided is a fluid reagent prepared by a method comprising the step of incubating together for a predetermined period of time, in a medium, a first population of cultured cells that is at least 80% mesenchymal cells (MSCs) and a second population of cells in the medium that is at least 80% VSELs, followed the step of storing the fluid reagent as it occurs following the incubating for the predetermined period of time. Also provided is the above fluid reagent that is cell free. Also provides is the above fluid reagent that is not cell free. Moreover, what is also provided is the above fluid reagent, that is capable of stimulating confluence of human dermal fibroblasts, capable of stimulating closing of a gap in an assay of human dermal fibroblasts, and capable of stimulating expression of collagen expression by the human dermal fibroblasts.

In yet another aspect, what is provided is the above fluid reagent, that is: (a) Capable of stimulating confluence of human dermal fibroblasts; (b) capable of stimulating closing of a gap in an assay of human dermal fibroblasts as determined by GAP assay method of Example 1; and (c) Capable of stimulating expression of pro-collagen type-1 expression by the human dermal fibroblasts. What is also provided is the above fluid reagent that is: (a) Capable of stimulating confluence of human dermal fibroblasts; (b) Capable of stimulating closing of a gap in an assay of human dermal fibroblasts as determined by GAP assay method of Example 1 wherein the gap at time=zero hours is at least 100 micrometers (μm) wide and wherein the number of cells in the gap at time=greater than 24 hours is greater than 150 cells; and (c) Capable of stimulating expression of pro-collagen type-1 expression by the human dermal fibroblasts.

Moreover, what is also provided is above fluid reagent that is: (a) Capable of stimulating confluence of human dermal fibroblasts; (b) Capable of stimulating closing of a gap in an assay of human dermal fibroblasts as determined by GAP assay method of Example 1 wherein the gap at time=zero hours is at about 100 micrometers (μm) wide and wherein the number of cells in the gap at time=greater than 24 hours is greater than 200 cells; and (c) Capable of stimulating expression of pro-collagen type-1 expression by the human dermal fibroblasts. In yet another aspect, what is provided is above fluid reagent in combination with a pharmaceutically acceptable excipient. In embodiments, the excipient can promote absorption of peptides into the skin, or can promote absorption of one or more of glycopeptides, glycolipids, oligosaccharides, lipids, polypeptides, nucleic acids, and the like, into the skin.

In another aspect, what is provided is the above fluid reagent, further comprising separating the fluid reagent into a first fraction that comprises one or more peptides that are: (a) Capable of stimulating confluence of human dermal fibroblasts; (b) Capable of stimulating closing of a gap in an assay of human dermal fibroblasts as determined by GAP assay method of Example 1 wherein the gap at time=zero hours is at about 100 micrometers (μm) wide and wherein the number of cells in the gap at time=greater than 24 hours is greater than 200 cells; and (c) Capable of stimulating expression of pro-collagen type-1 expression by the human dermal fibroblasts; a second fraction that does not contain said one or more peptides, and discarding the second fraction.

In methods embodiments, what is provided is a method for stimulating growth of at least one human dermal fibroblast, comprising contacting a growth stimulatory amount of the above fluid reagent to: (a) The skin of a subject, wherein the contacting is topical; or (b) To a wound of a subject. Also provided is above method, wherein the subject is a human subject. Also provided is above method, wherein the skin comprises one or more of age-induced wrinkles, ultraviolet light-induced wrinkles, subdermal ultraviolet light-induced damage, and scar tissue. Also provided is above method that results in one or more of reduction of age-induced wrinkles, reduction of ultraviolet light-induced wrinkles, reduction of subdermal ultraviolet light-induced damage, reduction in scar tissue, and increase in dermal thickness. Also provided is above method, wherein the above fluid reagent further comprises an excipient that facilitates absorption of peptides by the skin. Also provided is the above method, wherein the subject is mammalian. Also provided is the above method, wherein the subject is human, a veterinary subject, or an agricultural livestock subject.

In composition embodiments, what is provided is a composition comprising cultured cells in a medium, further comprising a first population of cultured cells in the medium that is at least 80% mature mesenchymal stem cells (MSCs) and a second population of cells in the medium that is at least 80% small cells, wherein the small cells are recently divided MSCs or progenitors of MSCs. Also provided is above composition, wherein each of the mature MSCs is CD105+, CD90+, CD73+, CD44+. In another composition embodiment, what is provided is a fluid reagent prepared by a method comprising the step of: incubating together for a predetermined period of time, in a medium, a first population of cultured cells that is at least 80% mesenchymal cells (MSCs) and a second population of cells in the medium that is at least 80% small cells that are recently divided MSCs or progenitors of MSCs, followed the step of: storing the fluid reagent as it occurs following the incubating for the predetermined period of time. Also provided is the above fluid reagent, wherein the storing is in a cool environment, wherein the storing is stored frozen, or wherein the storing is stored in a dried or dessicated state.

The disclosure provides a method for co-culturing mesenchymal cells (MSCs) and very small embryonic-like stem cells (VSELs), comprising: contacting at least one MSC to a first culture medium, contacting at least one VSEL to a second culture medium, or contacting at least one MSCs and also contacting at least one VSELs to an identical medium (third medium), wherein the first medium, second medium, or third medium, do not contain exogenous growth factors, and incubating the cells. What is also embraced is the above method, wherein the MSCs and VSELs are separated by a semi-permeable membrane or by a microfluidics channel, as well as the above method wherein the MSCs and VSELs are not separated by a semi-membrane or by a microfluidics channel.

Moreover, what is encompassed is the above method, wherein the MSCs are capable of forming processes or filopodia, or wherein the VSELs are capable of forming processes or filopodia, and wherein at least one MSC process or filopodia contacts the VSELs, or wherein at least one VSEL process or filopodia contact the MSCs. What is also disclosed, is the above method, wherein the first medium has a composition that is identical to the composition of the second medium.

Furthermore, what is provided is the above method, wherein the MSCs stimulate growth of the VSELs, wherein VSELs stimulate growth of the MSCs, or wherein MSCs stimulate growth of VSELs and the VSELs stimulate growth of the MSCs. In an exemplary embodiment, what is provided is the above method, wherein the MSCs stimulate growth of VSELs: by way of at least one soluble factor, by a signal requiring direct contact of MSCs to VSELs, or by both at least one soluble factor and by direct contact, or wherein the VSELs stimulate growth of MSCs: by way of at least one soluble factor, by at least one signal requiring direct contact of VSELs to MSCs, or by both at least one soluble factor and by direct contact. Also provided, is the above method, further comprising isolating or purifying the MSCs away from the VSELs, as well as the above method, further comprising isolating or purifying the VSELs away from the MSCs. Yet another embodiment, is a composition that comprises one or more culture media that result from, or prepared by, one or more of the above-disclosed methods. Also embraced is the a method for administering the above-disclosed composition of to a subject, comprising transferring the composition from a container to a subject by way of a technique involving an injection, transfusion, surgery, direct application, or topical application.

What is also embraced is the above method, and the above composition, where the cells are of human origin, where the cells are of non-human origin, where the cells are of murine origin, or where the cells have been genetically engineered.

In embodiments, what is provided is a method for co-culturing at least one VSEL and at least one cell that is not a VSEL, comprising contacting at least one non-VSEL to a first culture medium, contacting at least one VSEL to a second culture medium, or contacting at least one non-VSEL and also contacting at least one VSELs to an identical medium (third medium), and incubating the cells. Also provided is the above method, where the first culture medium, second culture medium, or third medium, comprise serum that contains endogenous growth factors. In addition, what is encompassed is the above method, wherein none of the culture media contain endogenous growth factors.

In another aspect, what is embraced is the above method, wherein none of the culture media contain endogenous growth factors, and wherein at least one of the culture media contains an exogenous growth factor. Also disclosed, is the above method, wherein none of the culture media contains endogenous culture growth factors, wherein none of the culture media contains an exogenous growth factor, and wherein at least one of the culture media contains a growth factor mimetic. Moreover, what is included is the above method, wherein the non-VSEL does not comprise sarcoma cells. Also provided is a composition produced by the above methods.

### BRIEF DESCRIPTIONS OF THE FIGURES

FIG. 1. The figure shows growth rates and cell confluence of human dermal fibroblasts (CCL-110 cells).

FIG. 2. The figure shows growth rates and cell confluence of human dermal fibroblasts (SCRC-1041 cells).

FIG. 3. Area under the curve analysis of the growth (CCL-110 cells). Area under the curve is solved by calculus integrals to quantify the amount of space under a curve for an X/Y plot.

FIG. 4. Area under the curve analysis of the growth (SCRC-1041 cells). Area under the curve is solved by calculus integrals to quantify the amount of space under a curve for an X/Y plot.

FIG. 5. Collagen production from human dermal fibroblasts (CCL-110 cells).

FIG. 6. Collagen production from human dermal fibroblasts (SRC-1041 cells).

FIG. 7. GAP assay (0 min).

FIG. 8. GAP assay (288 min).

FIG. 9. GAP assay (576 min).

FIG. 10. GAP assay (864 min).

FIG. 11. GAP assay (1152 min).

FIG. 12. GAP assay (1440 min).

FIG. 13. The figure shows analysis of Gap Assay results for human dermal fibroblasts (CCL-110 cells).

FIG. 14. The figure shows analysis of Gap Assay results for human dermal fibroblasts (SCRC-1041 cells).

FIG. 15. The figure is a photograph of GAP results from human mesenchymal stem cells.

FIG. 16. The figure shows growing human bone marrow-derived stem cells. The four arrows that are superimposed on the photograph point to dividing stem cells.

FIG. 17. The figure shows a photograph of stem cell culture. The long, dashed arrows indicate Mesenchymal Stem Cells, and the short solid arrows indicate small stem cells.

FIG. 18. The figure shows stem cells and MSC Division. The following time sequence of MSC cell division is shown: FIG. 18A (0 min), FIG. 18B (33.5 min), FIG. 18C (45.5 min), FIG. 18D (82 min), FIG. 18 E 85 min), FIG. 18F (94 min), FIG. 18G (118 min), and FIG. 18H (218 min).

FIG. 19. The figure shows stem cells, MSCs (thick black arrows) and Small Stem Cells (thin black arrows).

FIG. 20. MSC contacts with other MSCs and with Small Stem Cells (VSELs). FIG. 20A. Photo taken at 170 minutes. FIG. 20B. Photo taken at 248 minutes. FIG. 20C. Photo taken at 314 minutes. FIG. 20D. Photo taken at 378 minutes. FIG. 20E. Photo taken at 470 minutes. FIG. 20F. Photo taken at 561 minutes.

FIG. 21. MSCs of the present disclosure are positive for CD105, CD90, CD73, and CD44.

### DETAILED DESCRIPTION OF THE DISCLOSURE

The present disclosure provides a purified or isolated population of one type of cell. In addition, what is provided is a purified or isolated population of a first type of cell in combination with a purified or isolated population of a second type of cell. In another aspect, the disclosure provides, in combination, a purified or isolated population of a first type of cell, a second type of cell, and a third type of cell. In aspects, the purified or isolated population is at least 50% homogeneous, at least 60%, at least 70%, at least 80%, at least 90%, at least 95%, at least 99%, or at least 100% homogeneous. Homogeneity can be determined, for example, by expression of markers, by morphology of the cell in question, by morphology of a three dimensional structure taken by a cluster of the cells, e.g., spherical morphology, or by ability of the cell to respond to a specific agonist or antagonist, for example, ability of the cell to respond to an agonist that provokes differentiation, activation, or maturation. Marker expression includes expression that reflects levels of messenger RNA (mRNA), levels of polypeptide, or change in subcellular location of a given marker, e.g., nuclear location versus cytoplasmic location. Techniques and equipment for measuring expression, and for identifying cells, include flow cytometry, histology, gene arrays, and reagents such as antibodies, enzyme-linked antibodies, fluorescent antibodies, polymerase chain reaction (PCR), and the like. Guidance on flow cytometry is available (see, e.g., BD Biosciences, San Jose, Calif. (December 2007) BD FACSAria II User's Guide, part no. 643245, Rev.A (344 pages)).

Equipment, methods, and media, for cell culture are provided. Two types of cells can be cultured in separate containers, but with communication by way of a permeable membrane, where the membrane allows passage of, for example, growth factors from the first culture to the second culture, and/or from the second culture to the first culture (see, e.g., Lichtenberg et al (2005) Biomaterials 26:555-562). The membrane can have a cutoff size of at least 0.05 micrometers, 0.1, 0.2, 0.5, 1.0, 2.0, 5.0, 10.0, 20.0, at least 50.0 micrometers, and the like. Also, the membrane can have a cutoff size of less than 0.05 micrometers, 0.1, 0.2, 0.5, 1.0, 2.0, 5.0, 10.0, 20.0, less than 50.0 micrometers, and the like. In other aspects, the membrane can have a cutoff size that allows only passage of molecules with a molecular weight of under about 200 Daltons (D); 500D; 1000D; 2,000D; 5,000D; 10,000D; 20,000D; 50,000D; 100,000D; 200,000D; 500,000D; 1,000,000D; and so on. Also available, is a 2-compartment culture container, allowing a constant source of nutrients from a medium compartment, where the medium flows through a semi-permeable membrane to a cell compartment, thereby allowing an increased yield of cell, or of cell products, such as exosomes (see, e.g., Mitchell et al (2008) J. Immunol. Methods. 335:98-105). Two types of cells can also be cultured in a microfluidics device, where the two types of cells are separated by microgrooves. The two types of cells are separated, but the microgrooves allow processes to reach from one type of cell to the other (see, e.g., Taylor et al (2005) Nat. Methods. 2:599-605). Cell culture media, culture flasks, membranes, fermenters, flow cytometers, centrifugal elutriation devices, and cell culture bioreactors, are available, e.g., from BD Biosciences, San Jose, Calif.; Beckman Coulter, Brea, Calif.; New Brunswick Scientific, Enfield, Conn.; and Integra Biosciences AG, Zizers, Switzerland. Instruments for confocal microscopy are available (e.g., Carl Zeiss, Thornwood, N.Y.).

### DEFINITIONS

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a derivative of GM-CSF, an antibody that stimulates GM-CSF receptor. An “antagonist,” as it relates to a relationship between a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

“Conservatively modified variants” applies to both amino acid and nucleic acid sequences. With respect to particular nucleic acid sequences, a conservatively modified variant refers to nucleic acids encoding identical amino acid sequences, or amino acid sequences that have one or more conservative substitutions. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).

### (1) Hydrophobic: Norleucine, Ile, Val, Leu, Phe, Cys, Met;

(2) Neutral hydrophilic: Cys, Ser, Thr;

### (3) Acidic: Asp, Glu;

### (4) Basic: Asn, Gin, His, Lys, Arg;

(5) Residues that influence chain orientation: Gly, Pro;

### (6) Aromatic: Trp, Tyr, Phe; and

(7) Small amino acids: Gly, Ala, Ser.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. “Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual. See, e.g., U.S. Pat. No. 5,888,530 issued to Netti, et al, which is incorporated herein by reference.

An “extracellular fluid” encompasses, e.g., serum, plasma, blood, interstitial fluid, cerebrospinal fluid, secreted fluids, lymph, bile, sweat, fecal matter, and urine. An “extracellular fluid” can comprise a colloid or a suspension, e.g., whole blood or coagulated blood.

“Gene” refers to a nucleic acid sequence encoding an oligopeptide or polypeptide. The oligopeptide or polypeptide can be biologically active, antigenically active, biologically inactive, or antigenically inactive, and the like. The term gene encompasses, e.g., the sum of the open reading frames (ORFs) encoding a specific oligopeptide or polypeptide; the sum of the ORFs plus the nucleic acids encoding introns; the sum of the ORFs and the operably linked promoter(s); the sum of the ORFS and the operably linked promoter(s) and any introns; the sum of the ORFS and the operably linked promoter(s), intron(s), and promoter(s), and other regulatory elements, such as enhancer(s). In certain embodiments, “gene” encompasses any sequences required in cis for regulating expression of the gene. The term gene can also refer to a nucleic acid that encodes a peptide encompassing an antigen or an antigenically active fragment of a peptide, oligopeptide, polypeptide, or protein. The term gene does not necessarily imply that the encoded peptide or protein has any biological activity, or even that the peptide or protein is antigenically active. A nucleic acid sequence encoding a non-expressable sequence is generally considered a pseudogene. The term gene also encompasses nucleic acid sequences encoding a ribonucleic acid such as rRNA, tRNA, or a ribozyme.

“Growth factor” encompasses factors that stimulate growth, where this encompasses polypeptide and oligopeptide growth factors, polypeptide and oligopeptide hormones, and hormones that are not polypeptides. “Growth factor” also encompasses mutated polypeptide growth factors, or chemically modified small molecule growth factors, that may occur naturally and that have growth factor stimulating ability. Although nutrients such as carbohydrates, fats, minerals, and vitamins are required for growth, these are generally not considered to be growth factors. Polypeptides, peptides, chemicals, small molecules, and compositions that are mimetics of naturally occurring growth factors, but that are not likely to arise naturally, are classified as mimetics.

A composition that is “labeled” is detectable, by spectroscopic, photochemical, biochemical, immunochemical, isotopic, or chemical methods. For example, labels include radioactive isotopes of phosphorous, iodine, sulfur, carbon, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

“Ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity (the same name), or it may have a different identity (a different name), as the first cell. A ligand or receptor may be entirely intracellular, that is, it may reside in the cytosol, nucleus, or in some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

### EXAMPLES

### Example One

### Gap Assay

Guidance for conducting gap assays, and for the analysis of data from gap assays, is available without impying any limitation, from Liang et al (2007) 2:329-333, which is incorporated herein by reference in its entirety.

For conducting GAP assay, a gap can be created in a group of cells in cell culture, where the gap is about 0.02 mm, about 0.04 mm, about 0.06 mm, about 0.08 mm, about 0.1 mm, about 0.2 mm, about 0.3 mm, about 0.4 mm, 0.5 mm, 0.6 mm, 0.7 mm, about 0.8 mm, 0.9 mm, about 1.1 mm, about 1.2 mm, about 1.3 mm, 1.4 mm, 1.5 mm, about 1.6 mm, 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, about 3.0 mm, and so on. In this context, the term “about” means greater than the preceding value, and lesser than the succeeding values. What is encompassed is all ranges of the above values, such as: gap from about 6.0 mm to about 10.0 mm. A preferred gap size is 0.10 millimeters (mm).

Number of cells entering the gap, as assessed by the GAP assay, can be, for example, 1 cell, 2 cells, 3 cells, 4 cells, 5 cells, 6 cells, 7 cells, 8 cells, 9 cells, 10 cells, 11 cells, 12 cells, 13 cells, 14 cells, 15 cells, 16 cells, 17 cells, 18 cells, 19 cells, 20 cells, and so on, and any additive combination of these, and any range of the additive combinations, such as 20-40 cells, 40-60 cells, 60-80 cells, 80-100 cells, 100-120 cells, 120-140 cells, 140-180 cells, 180-200 cells, and the like. To provide some non-limiting examples, the present disclosure provides a medium that results in the complete filling of a 2.0 mm GAP, where the complete filling is with 180-200 cells, in a given time frame. The time frame can be, for example, about 200 min, about 250 min, about 300 min, about 350 min, about 400 min, about 450 min, about 500 min, about 550 min, about 600 min, about 650 min, about 700 min, about 750 min, about 800 min, about 850 min, about 900 min, about 950 min, about 1,000 min, about 1,100 min, about 1,200 min, about 1,300 min, about 1,400 min, about 1,500 min, about 1,600 min, about 1,700 min, about 1,800 min, about 1,900 min, about 2,000 min, and the like. In this context, “about” means a range that is greater than the previous value, to less than the subsequent value.

Also, the time frame can be somewhat arbitrary, and can refer to time to confluence. For example, the present disclosure provides a medium that results in the complete filling of a 2.0 mm GAP, where the complete filling is with 180-200 cells, in a time frame of 1,600 minutes. Provided is a medium that results in a cell count of at least 150 cells, at least 160 cells, at least 170 cells, at least 180 cells, at least 190 cells, at least 200 cells, at least 210 cells, at least 220 cells, at least 230 cells, at least 240 cells, at least 250 cells, at least 260 cells, at least 270 cells, at least 280 cells, and the like, where the time frame is “at least 24 hours,” and where a comparator medium (or a control medium) gives fewer cells. The fewer cells that results from the comparator medium can be, for example, fewer than 220 cells, fewer than 210 cells, fewer than 200 cells, fewer than 190 cells, fewer than 180 cells, fewer than 170 cells, fewer than 160 cells, fewer than 150 cells, fewer than 140 cells, fewer than 130 cells, fewer than 120 cells, fewer than 110 cells, fewer than 100 cells, fewer than 90 cells, fewer than 80 cells, fewer than 70 cells, fewer than 60 cells, fewer than 50 cells, fewer than 40 cells, and so on.

Mesenchymal stem cells (MSCs) are co-cultured with very small embryoid-like stem cells (VSELs). MSCs have a diameter of about 8 micrometers, while VSELs have a diameter of about 2-3 micrometers. VSELs are unique, in that they do not exclude trypan blue, due to pores in the membrane that allow passage of trypan blue. They have relatively little cytoplasm, and some have very few mitochondria. The present disclosure provides co-culture of MSCs and VSELs in the absence of added growth factors. During co-culture, the VSELs grow faster than MSCs.

### Identifying VSELs

VSELs can be purified by conventional techniques of cell biology, where VSELs can be purified and identified by techniques based on the very small size of VSELs, large nucleus surrounded by a narrow rim of cytoplasm, open-type chromatin (euchromatin), high ratio of nuclear-to-cytoplasm, and by expression markers. Murine VSEL expression markers include SSEA-1, Oct-4, Nanog, Rex-1, Dppa3, and Rif-1. Human VSEL expression markers include SSEA-4, Oct-4, and Nanog. VSELs also express several markers of primordial germ cells (PGCs). For example, murine VSELs express placental form of alkaline phosphatase, Stella, Fragilis, Nobox, Hdac6, and CXCR4. One study, for example, isolated murine VSELs on the basis of Sca-1+; Lin−; CD45−, expression (Zuba-Surma et al (2008) Cytometry 73A:1116-1127). Nucleic acid probes, as well as antibodies, for these markers are available, or can be designed with sequence information available in the nucleotide database of National Center for Biotechnology Information (NCBI), National Institutes of Health (NIH). VSELs are also distinguished by their high migratory capability towards gradients of SDF-1, hepatocyte growth factor (HGF), and leukemia inhibitory factor (LIF). Pluripotency of VSELs can be shown by their ability to differentiate into cellular lineages from all three germ layers. Methods and reagents for identifying and isolating VSELs are disclosed (see, e.g., US 20120021482 of Zuba-Surma et al, US 20120045758 of Kucia et al, which are hereby incorporated by reference in its entirety). VSELs are related to, and may be (in part or in entirety) the same as spore-like cells (see, e.g., 7,964,394; 7,575,921; 7,560,275; 7,319,035; 7,060,492, each issued to Vacanti et al, each of which are incorporated by reference in their entirety).

VSELs promote or stimulate growth of MSCs. Growth stimulation can be manifest in terms of greater cell number, or in terms of greater amount of protein content per cell. Promotion of growth by VSELs can be via secretion by VSEL of growth factors with binding of the growth factors to MSCs, or via VSEL's expression of a membrane-bound growth factor that binds to a membrane-bound receptor of MSCs. The growth factors can directly stimulate growth of MSCs or, alternatively, can inhibit a growth dampening mechanism that exists in MSCs.

MSCs stimulate greater growth of VSELs. Growth stimulation can be manifest in terms of greater cell number, or in terms of greater amount of protein content per cell. Promotion of growth by MSCs can be via secretion by MSCs of growth factors with binding of the growth factors to VSELs, or via MSC's expression of a membrane-bound growth factor that binds to a membrane-bound receptor of VSELs. The growth factors can directly stimulate growth of VSELs or, alternatively, can inhibit a growth dampening mechanism that exists in VSELs. As stated above, In the present disclosure, the abbreviation “MSC” refers only to mesenchymal stem cells.

In co-culture, on occasion VSEL and MSC contact each other, by way of a process from the VSEL contacting a process of the MSC. The present disclosure provides methods for enhancing this contacting, as well as method for impairing this contacting. Enhancing the contacting can be accomplished by co-culturing cells at greater density. Impairing the contacting can be accomplished by culturing VSELs and MSCs in two different chambers, separated by a filter or by grooves in a microfluidic device, which allow exchange of factors secreted by the cells.

What is provided is a method for stimulating growth of MSCs by co-culturing MSCs with VSELs, in absence of exogenous growth factors. Also, the disclosure provides a method for stimulating growth of VSELs by co-culturing MSCs with VSELs, in absence of exogenous growth factors. Conditions for eliminating the effective concentration of one or more growth factors include, e.g., supplementing medium with antagonistic antibodies to growth factors, or blocking antibodies to growth factor receptors.

Growth factors, mimetics of growth factors, and agonistic antibodies that stimulate the growth factor receptor, include the following. For each of these, the present disclosure provides a serum or other biological fluid that contains one or more of these. For each of these, the present disclosure provides a serum or other biological fluid that has been selectively depleted in one or more of these, for example, depletion by antibody capture. For each of these, the present disclosure provides one or more exogenous molecules, for example, a purified and homogenous molecule.

epidermal growth factor;

endothelial growth factor;

platelet-derived growth factor;

insulin-like growth factor-1 (IGF-1);

insulin-like growth factor-2 (IGF-2);

granulocyte colony stimulating factor (G-CSF),

granulocyte macrophage colony stimulating factor (GM-CSF);

fibroblast growth factor (FGF);

nerve growth factor (NGF);

vascular endothelial growth factor (VEGF);

interleukin-1 (IL-1), IL-2, IL-3, IL-4, IL-5, IL-6, IL-7, and other cytokines.

Soluble receptors can also provide a blocking function against a growth factor or against a ligand. Growth factors, cytokines, and antibodies, are available from, e.g., Life Technologies, Carlsbad, Calif.; Sigma-Aldrich, St. Louis, Mo.; Thermo Fisher Scientific, Rockford, Ill.; Antibodies, Inc., Miltenyi Biotech, Koln, Germany; Davis, Calif., R & D Systems, Minneapolis, Minn. Culture media, related reagents, and cell lines, including, CCL-110 and SCRC-1041, are available, e.g., from American Type Culture Collection (ATCC), Manassas, Va.

The present disclosure provides a medium that lacks one of the following, that is, that lacks epidermal growth factor; that lacks endothelial growth factor; that lacks platelet-derived growth factor; that lacks insulin-like growth factor-1 (IGF-1) or IGF-2; that lacks granulocyte colony stimulating factor (G-CSF), that lacks granulocyte macrophage colony stimulating factor (GM-CSF), that lacks fibroblast growth factor (FGF), that lacks nerve growth factor (NGF), or that lacks vascular endothelial growth factor (VEGF). Moroever, what is provided is a medium that lacks all of the above factors.

### Exclusionary Embodiments

Also provided is a medium that lacks two of the following, that is, that lacks epidermal growth factor and endothelial growth factor; epidermal growth factor and platelet-derived growth factor; epidermal growth factor and insulin-like growth factor-1 (IGF-1) or IGF-2; epidermal growth factor and granulocyte colony stimulating factor (G-CSF); epidermal growth factor and granulocyte macrophage colony stimulating factor (GM-CSF), epidermal growth factor and fibroblast growth factor (FGF), epidermal growth factor and nerve growth factor (NGF), epidermal growth factor and vascular endothelial growth factor (VEGF). What is provided is a medium where one or more of the above growth factors has not been added, that is, where one or more of the above growth factors has not been added as an exogenous supplement. Thus, what is provided are exclusionary embodiments, that exclude reagents, media, and methods, where what is left out or what is omitted is one or more exogenous growth factors.

Other combinations of two factors that, in some embodiments, are excluded include: endothelial growth factor and platelet-derived growth factor; endothelial growth factor and insulin-like growth factor-1 (IGF-1) or IGF-2; endothelial growth factor and granulocyte colony stimulating factor (G-CSF), endothelial growth factor and granulocyte macrophage colony stimulating factor (GM-CSF), endothelial growth factor and fibroblast growth factor (FGF), endothelial growth factor and nerve growth factor (NGF), endothelial growth factor and vascular endothelial growth factor (VEGF).

Yet additional sets of two factors that are excluded from medium, in embodiments, are platelet-derived growth factor and insulin-like growth factor-1 (IGF-1) or IGF-2; platelet-derived growth factor and granulocyte colony stimulating factor (G-CSF), platelet-derived growth factor and granulocyte macrophage colony stimulating factor (GM-CSF), platelet-derived growth factor and fibroblast growth factor (FGF), platelet-derived growth factor and nerve growth factor (NGF), platelet-derived growth factor and vascular endothelial growth factor (VEGF).

In embodiments, the following groups of factors are excluded in medium, that is, insulin-like growth factor-1 (IGF-1) or IGF-2 and granulocyte colony stimulating factor (G-CSF), insulin-like growth factor-1 (IGF-1) or IGF-2 and granulocyte macrophage colony stimulating factor (GM-CSF), insulin-like growth factor-1 (IGF-1) or IGF-2 and fibroblast growth factor (FGF), insulin-like growth factor-1 (IGF-1) or IGF-2 and nerve growth factor (NGF), insulin-like growth factor-1 (IGF-1) or IGF-2 and vascular endothelial growth factor (VEGF).

In alternate embodiments, what is provided is medium that lacks the following two factors, that is, granulocyte colony stimulating factor (G-CSF) and granulocyte macrophage colony stimulating factor (GM-CSF), granulocyte colony stimulating factor (G-CSF) and fibroblast growth factor (FGF), granulocyte colony stimulating factor (G-CSF) and nerve growth factor (NGF), granulocyte colony stimulating factor (G-CSF) and vascular endothelial growth factor (VEGF).

In further embodiments, what is provided is medium that lacks the following two factors, that is, granulocyte macrophage colony stimulating factor (GM-CSF) and fibroblast growth factor (FGF), granulocyte macrophage colony stimulating factor (GM-CSF) and nerve growth factor (NGF), granulocyte macrophage colony stimulating factor (GM-CSF) and vascular endothelial growth factor (VEGF).

What is also embraced, is medium lacking in the following two factors, that is, fibroblast growth factor (FGF) and nerve growth factor (NGF), fibroblast growth factor (FGF) and vascular endothelial growth factor (VEGF), as well as the combination of nerve growth factor (NGF) and vascular endothelial growth factor (VEGF).

In embodiments, what is provided is medium lacking in any three, any four, any five, any six, any seven, any eight, and the like, of the above individual factors.

In inclusory embodiments, what is provided is medium that includes one or more of epidermal growth factor; endothelial growth factor; platelet-derived growth factor; insulin-like growth factor-1 (IGF-1) or IGF-2; granulocyte colony stimulating factor (G-CSF), granulocyte macrophage colony stimulating factor (GM-CSF), fibroblast growth factor (FGF), nerve growth factor (NGF), or vascular endothelial growth factor (VEGF). What can be included, for example, are the specific pairs identified above in the disclosure of excluded pairs.

### Small Molecule Agonists of Growth Factor Receptors and Mimetics of Growth Factors

The disclosure provides serum-free medium, and methods of use, where exogenous growth factors are not used or are excluded, and where the serum-free medium is supplemented with a small molecule agonist of growth factor receptor, or a mimetic of the growth factor. The present disclosure provides small molecule mimetics of growth factors, including fibroblast growth factor (FGF), platelet-derived growth factor, vascular endothelial growth factor (VEGF), hematopoietic growth factors, and a variety of other growth factors where the mimetics are agonists to the respective receptor (see, e.g., Anderson et al (2005) J. Neurochem. 95:570-583; Lin et al (2007) Growth Factors. 25:87-93; Leslie-Barbick et al (2011) Biomaterials. 32:5782-5789; Dudar et al (2008) Am. J. Physiol. Gastrointest. Liver Physiol. 295:G374-G381; 20080096796 of Saffell; 20110003851 of Lin et al; and 20060127404 of Huang et al, each of which hereby incorporated by reference in its entirety. Also provided are agonistic antibodies that stimulate growth factor receptor (see, e.g., Bugelski et al (2008) J. Biotechnol. 134:171-180; and 20060127404 of Huang et al, which are incorporated by reference in their entirety).

In another aspect, the disclosure provides a first composition that comprises one or soluble small molecules, one or more soluble macromolecules, one or more membrane-bound ligands, and any combination thereof, derived from MSCs, which stimulates growth of VSELs. Furthermore, the disclosure supplies a second composition that comprises one or soluble small molecules, one or more soluble macromolecules, one or more membrane-bound ligands, and any combination thereof, derived from VSELs, which stimulates growth of MSCs. Also, the disclosure provides a combination of the first composition and the second composition, where the combination can stimulate growth of VSELs, stimulate growth of MSCs, or stimulate growth of both VSELs and MSCs.

### Co-Culturing Embodiments

In embodiments, what is provided is method for in vitro co-culture of VSELs and mesenchymal stromal cells, VSELs and mesenchymal stem cells, VSELs and at least one cell that is not a mesenchymal stromal cell, VSELs and at least one cell that is not a mesenchymal stem cell, VSELs and at least one cell that is not a cancerous cell, VSELs and at least one cell that is not a transformed cell, VSELs and at least one cell that is not a sarcoma cell, VSELs and at least one other type of cell that does not comprises a feeder layer, VSELs and at least one cell of human origin; human VSELs and at least one other type of cell that is of human origin, human VSELs and at least one other type of cell that is of human origin where cells of non-human origin are not included. What is also provided is a composition that comprises a mixture of the above-identified cells that has been co-cultured, a composition that comprises isolated VSELs prepared by the above co-culture, a composition that comprises isolated mesenchymal stromal cells prepared by the above co-culture, and a composition that comprises isolated mesenchymal stem cells prepared by the above co-culture.

### Mesodermal Stem Cells (MSCs), Methods, Reagents, and Markers

Methods, reagents, and markers, are available for the preparation, isolation, culture, storage, and identification of mammalian cells, including mesodermal stromal cells, mesodermal stem cells, and various kinds of stem cells. See, e.g., U.S. Pat. No. 8,105,791 issued to Lundgren-Akerlund; US 2011/0064701 of Young and Lucas, which are incorporated by reference in their entirety.

### High Confluence Values

High confluence values mean that there is more cell growth. High confluence also mean that there is more expression of products by cells, including but not limited to, products that are one or more of peptides, oligopeptides, polypeptides, small molecules, lipids, oligosaccharides, glycolipids, glycoproteins, nucleic acids, and combinations of these. Increased products associated with confluence include membrane-bound proteins that can be solubilized with, e.g., sodium cholate or with Triton X-100, and include membrane-associated proteins that can be dissociated with, e.g., treatment with salt such as 200 mM sodium chloride.

### Pharmaceutical Uses

Experiments of the present disclosure reveal that topical application of products expressed from cells resulted in thickening of the skin, apparently arising from increased expression in the skin of collagen. Topical application of products of the present disclosure results in one or more of increased collagen expression, reorganization or reduction of wrinkles, and reorganization or reduction of scars. Experiments of the present disclosure show that MDFc19 increases collagen expression, where this expression is greater than that obtained over SCM9 medium (Production Media), and without exposure to stem cells. Active and expanding dermal fibroblasts are better at reorganizing and healing damaged dermis, than those that are inactive and not expanding, and that are not receiving stem cell active peptides that are in MDFc19. A medium formulation that results in high values in a gap assay, results in the high value because the particular medium formulation excels in stimulating communication between MSCs and VSELs. Stimulated VSELs migrate extensively in flasks. Stimulated VSELs connect extensively with MSCs. Stimulated VSELs make more contacts with the MSCs than with each other. Stimulated VSELs, in dividing, become situated off of the flask surface, as do the MSCs, in order to divide, and then the stimulated cells come back to the surface and stick down, and help pull the two new cells apart. Media of the present disclosure that result in high confluence, and in high gap values, such as the MDFc19 media, are used for skin care indications. These indications include treatment of wrinkles, treatment of hyperpigmentation, treatment of ultraviolet light injury, and the like. Indications also include those that are not necessarily topical, such as treatment of surgery-induced wounds, as well as wounds that are not induced by surgery. MDFc19 can be used in preferable concentrations of 9% and of 12-15%. The present disclosure also provides MDFc19, in an excipient, at concentrations of 1%, 2%, 3%, 4%, 5%, 6%, 7%, 8%, 9%, 10%, 11%, 12%, 13%, 14%, 15%, 16%, 17%, 18%, 19%, 20%, and the like, as well as in ranges, such as, 4-5%, 5-6%, 6-7%, 7-8%, 8-9%, 9-10%, 10-11%, 11-12%, 12-13%, 13-14%, 14-15%, 15-16%, and the like.

### Excipients and Storage

Suitable excipients include sodium chloride, sodium phosphate, a buffer at roughly neutral pH, dimethyl sulfoxide (DMSO), olive oil, vegetable oil, and combinations thereof. DMSO and vegetable oils are agents that can promote the penetration of various reagents, small molecules, pharmaceuticals, and nutrients, into the skin. The fluids, reagents, solutions, and slurries of the present disclosure can be stored in a cool environment, such as at 4 degrees centigrade, stored frozen, stored desiccated, stored in a dried state, stored in a powdered state, stored with a preservative, stored with glycerol (5-20%), and the like.

Small Cells Other than VSELs

The present disclosure encompasses all of the recited embodiments, but where small MSCs are used instead of VSELs. This type of embodiment encompasses compositions and methods where small MSCs and VSELs are both present, as well as compositions and methods where small MSCs are present, but VSELs are not present. The small MSC can take the form, for example, of an immature MSC, a primitive stem cell, or a progenitor stem cell to the mature MSC, or a mature MSC that has recently divided. Cell division results in two daughter cells. The present disclosure also encompasses small cells that have not yet been identified. For example, what is further encompassed is a small stem cell that is not a VSEL and not a progenitor of a MSC and that is not a recently divided MSC.

### Gap Values

Guidance for conducting gap assays, and for the analysis of results from gap assays, is available. For the present disclosure, a gap with the starting gap size (width) of 100 micrometers (μM) was used. Starting gap size can be, for example, 80-120 μM, 85-115 μM, 90-110 μM, 95-105 μM, and the like. See, e.g., Liang et al (2007) Nature Protocols. 2:329-333, which is incorporated herein in its entirety, and which is attached herein as APPENDIX ONE.

### DETAILED DESCRIPTIONS OF THE FIGURES

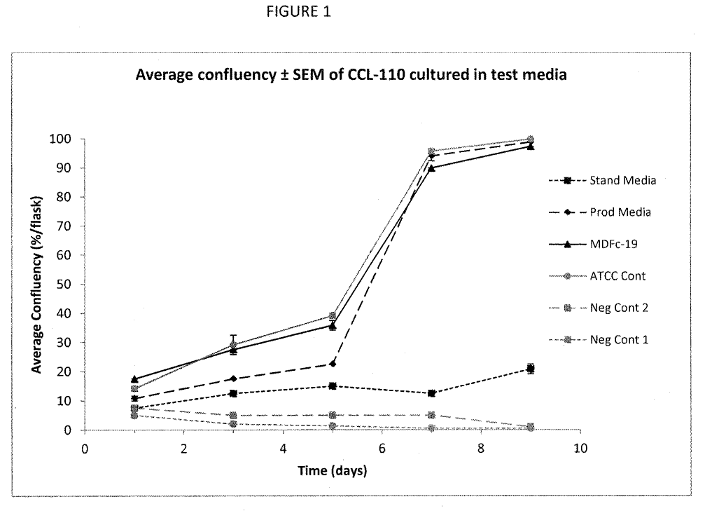

The following four figures (FIGS. 1-4) show use of human dermal fibroblasts to evaluate value of culturing with MDFc19 media developed from human adult stem cell cultures. This study compares growth of human dermal fibroblasts of 2 types (slow growing CCL-110 and rapid growing SCRC-1041) from the American Type Tissue Culture Collection in different cell culture media: a. Standard Media (CMRL+10% FBS), b. Production Media (SCM9+SCMG+10% FBS), c. MDFc-19 (Production Media used to grow human MSC's with the cells removed and used as skin care component), d. ATCC Control Media specifically for either CCL-110 cells or SCRC-1041 cells, e. Control Media 1 (CMRL+10% Human AB Serum), and e. Control Media 2 (SCM9+SCMG+10% Human AB Serum). CMRL-1066 medium, as provided by Sigma-Aldrich (St. Louis, Mo.), is shown in Table 1. The present disclosure is not to be limited to this supplier. CMRL-1066 is also available, e.g., from Life Technologies, Carlsbad, Calif.

The present disclosure provides a medium that provides: (Element 1) Superior confluence (greater confluency at, for example, at t=7 days, t=8 days, or t=9 days of culturing); (Element 2) Superior GAP assay results (greater number of cells filling the gap in the time frame of >24 hours); and (Element 3) Greater collagen expression; when compared to Negative Control Medium No. 1.

Also, the present disclosure provides a medium that provides: (Element 1) Superior confluence (greater confluency at, for example, at t=7 days, t=8 days, or t=9 days of culturing); (Element 2) Superior GAP assay results (greater number of cells filling the gap in the time frame of >24 hours); and (Element 3) Greater collagen expression; when compared to Negative Control Medium No. 2.

In other aspects, the present disclosure provides superior results with Element 1, than with a control medium, or superior results for Element 2 than with a control medium, or superior results with Element 3 than with a control medium, or superior results with Elements 1 and 2 than with a control medium, or superior results with Elements 1 and 3 than with a control medium, or superior results with Elements 2 and 3 than with a control medium.

Also, the present disclosure provides a medium that provides: (Element 1) Superior confluence (greater confluency at, for example, at t=7 days, t=8 days, or t=9 days of culturing); (Element 2) Superior GAP assay results (greater number of cells filling the gap in the time frame of >24 hours); and (Element 3) Greater collagen expression; when compared to Negative Control Medium No. 1.

MDFc-19 medium contains peptides resulting when both types of stem cells (MSCs and VSELs) were grown together, without standard MSC growth factors, except that fetal bovine serum (FBS) was added. These peptides contribute to stimulation of dermal fibroblasts, in the confluence assays and the GAP assays of the present disclosure.

FIG. 1 (CCL-110 Cells)

Analysis of growth rates and cell confluence of human dermal fibroblasts. The culture of the slow growing dermal fibroblasts CCL-110 cultured in the different media are plotted with average confluence percentages over days of culture. The Standard Media reached 15% confluence on Day 5 with 14% on Day 7 and 25% on Day 9. Both the ATCC Control Media and MDFc-19 media reached 30% confluence on Day 3, 40% on Day 5 and 93% on Day 7 with >95% confluence on Day 9 showing little difference in confluence between the ATCC Control Media and MDFc-19 media.

In contrast, the Production Media reached 15% confluence on Day 3 and 20% confluence on Day 5 and then reached 90% on Day 7 and 95% confluence on Day 9. Neither of the control 1 or 2 media resulted in any significant cell growth. Production Media was prepared from both types of stem cells (MSCs and VSELs) grown together. It was not the case that Production Media was prepared from both of these cells grown, but separated by a permeable membrane, or separated by any particular device.

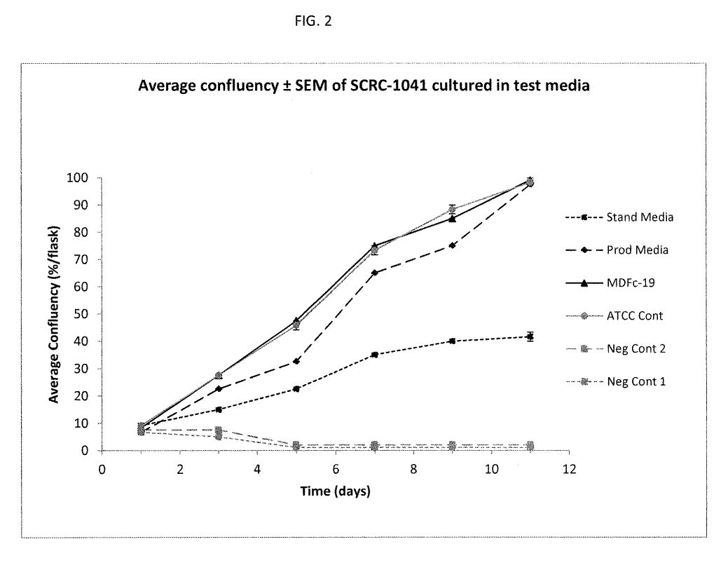

FIG. 2 (SCRC-1041 Cells)

A similar growth and confluence results were observed for the rapid growing SCRC-1041 dermal fibroblasts except that the growth was more gradual and ahead of the slower growing CCL-110 cells. The ATCC control Media and the MDFc-19 media had nearly identical results with 30% confluence on Day 3, 50% on Day 5, 75% on Day 7, 85% on Day 9, and >95% confluence on Day 11. The Production Media was slower in reaching the same level of confluence that the MDFc-19 and the ATCC Control Media, but did reach >95% on Day 11. The Standard Media was much slower that the other three media and only reached a 45% confluence level by Day 11 of culture. The Control Media 1 and 2 essentially showed no growth with loss of 10% confluence to 2% by Day 5 of culture.

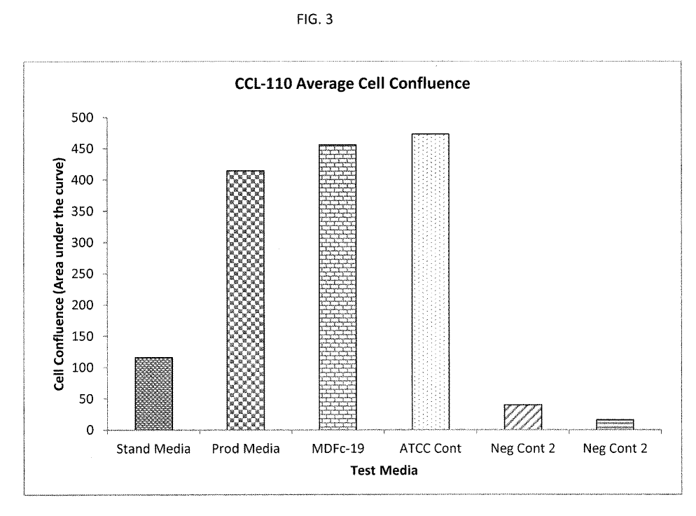

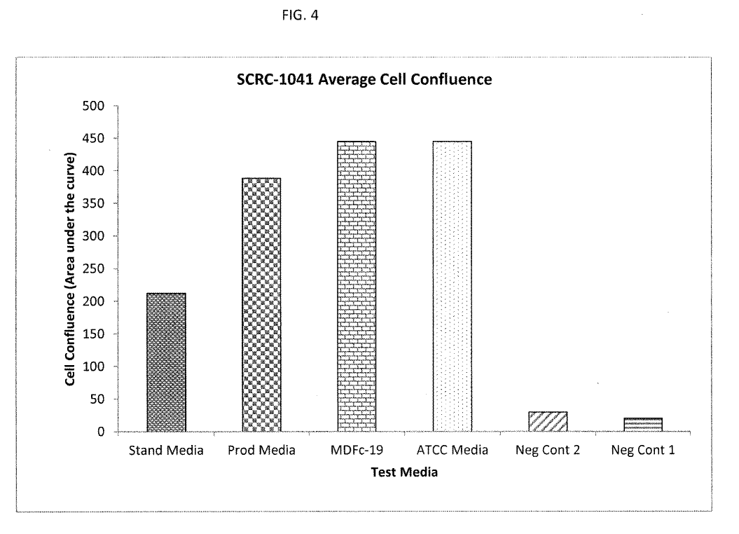

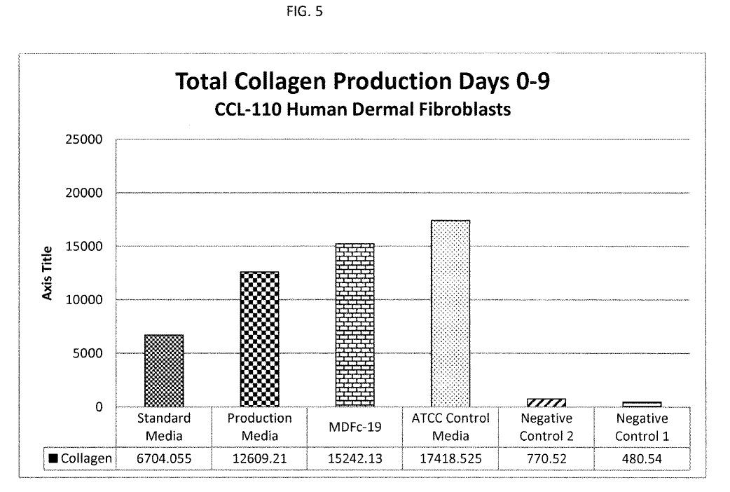

FIG. 3 (CCL-110 Cells) FIG. 4 (SCRC-1041 Cells)

FIG. 3 shows area under the curve analysis of the growth of the 2 types of dermal fibroblasts from the use of different culture media. The same results of confluence testing and growth as shown in FIG. 1, are shown in these analyses of the area under the curve for the different types of cell culture media used. Using both dermal fibroblast types of cells, the slow growing CCL-110 cells (FIG. 3) and the rapid growing SCRC-1041 cells (FIG. 4), the MDFc-19 cultures resulted in higher levels of cell confluence than the production media, with the only difference between these media the peptides released by the stem cells during the culture that produced MDFc-19 media. In terms of confluence there was little difference in the confluence achieved between the ATCC control media and the MDFC-19 media. The Standard Media growth was 30-50% of the level of the Production Media. The Control Media 2 and 1 resulted in very little growth of the dermal fibroblasts.

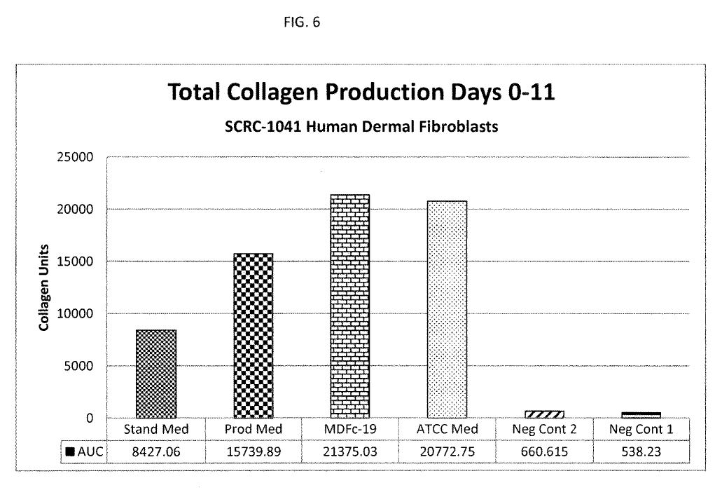

FIG. 5 (CCL-110 Cells) and FIG. 6 (SRC-1041 Cells)

FIG. 5 and FIG. 6 show collagen production from human dermal fibroblasts grown with different culture media including MDFc-19. During the same types of culture media protocol, media were collected over 9 days for the CCL-110 dermal fibroblasts (FIG. 5) and over 11 days for the SCRC-1041 dermal fibroblasts (FIG. 6) and assayed for Type 1 pro-collagen. Units of collagen are nanograms per mL.

The following concerns the analysis of collagen production. The pattern of collagen production for both the slow growing CCL-110 dermal fibroblasts (FIG. 5) and the fast growing SCRC-1041 dermal fibroblasts (FIG. 6) is somewhat similar with the different types of media (pattern with CCL-110 cells compared to pattern with SCRC-1041 cells). There is a clear increase in collagen production while the cells were cultured with MDFc-19 media in both the CCL-110 cells and the SCRC-1041 cells over the production media: a) CCL-110 cells with MDFc-19 Media=15,242 units while Production Media=12,609 units or a 36% increase in collagen production by using MDFc-19 and b) SCRC-1041 cells with MDFc-19 Media=21,375 units while the Production Media=15,740 units or another 36% increase in collagen production by MDFc-19. The Standard Media collagen production was only 53% of the Production Media for CCL-110 dermal cells while the Standard Media for SCRC-1041 dermal cells was only 54% of the Production Media. The only difference between the MDFc-19 media and the Production Media are the peptides produced by the stem cells while they were cultured. The Negative Control 1 and 2 produced very little collagen with both types of dermal fibroblasts.

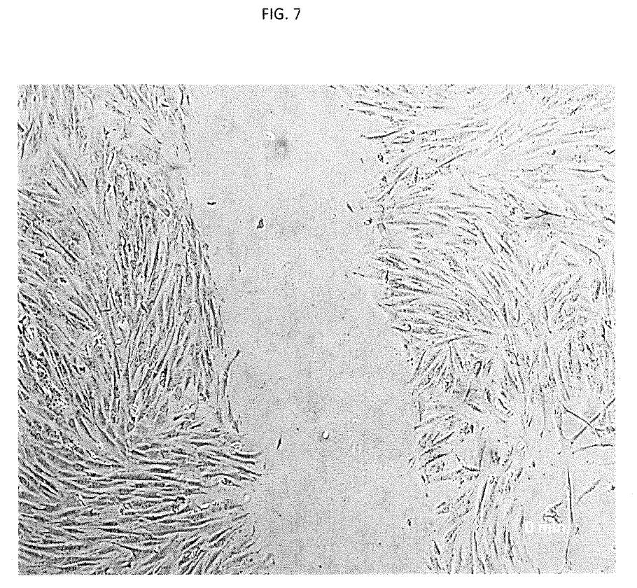

FIGS. 7-12 (Time Course)

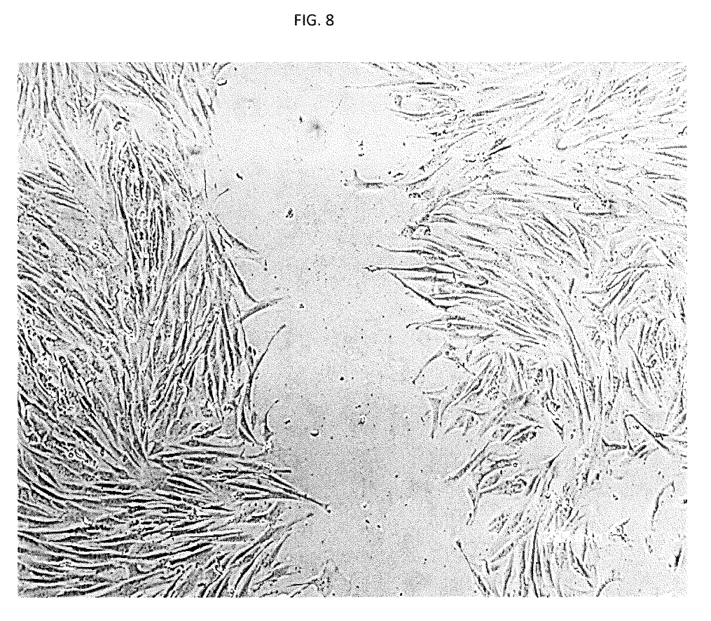

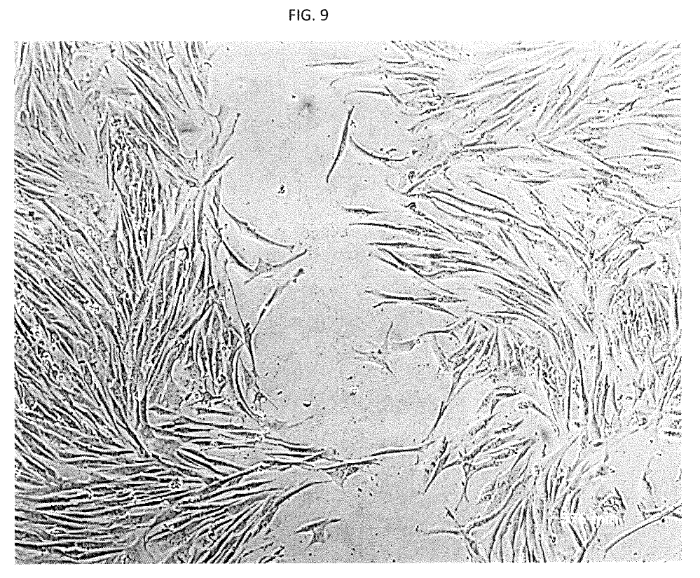

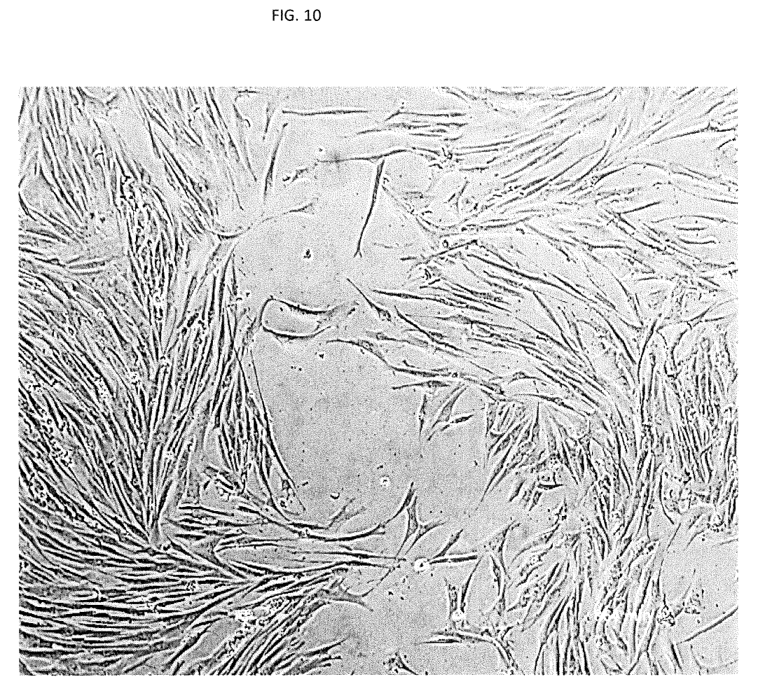

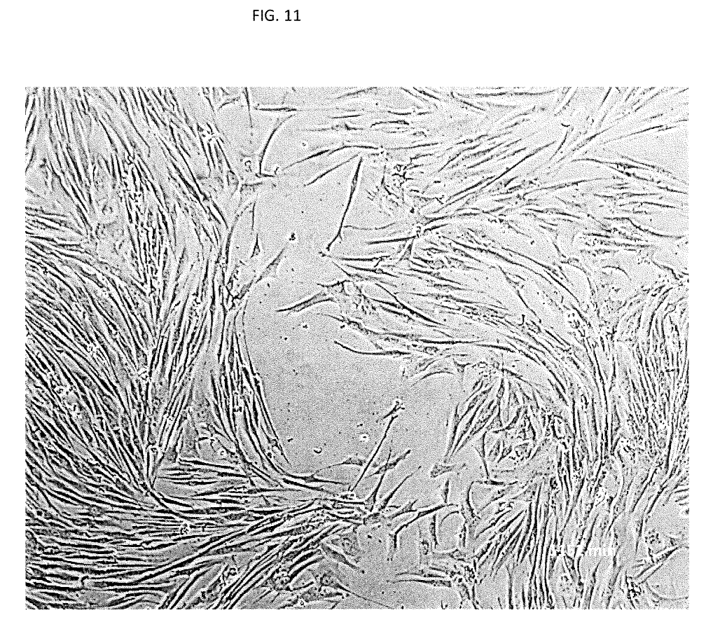

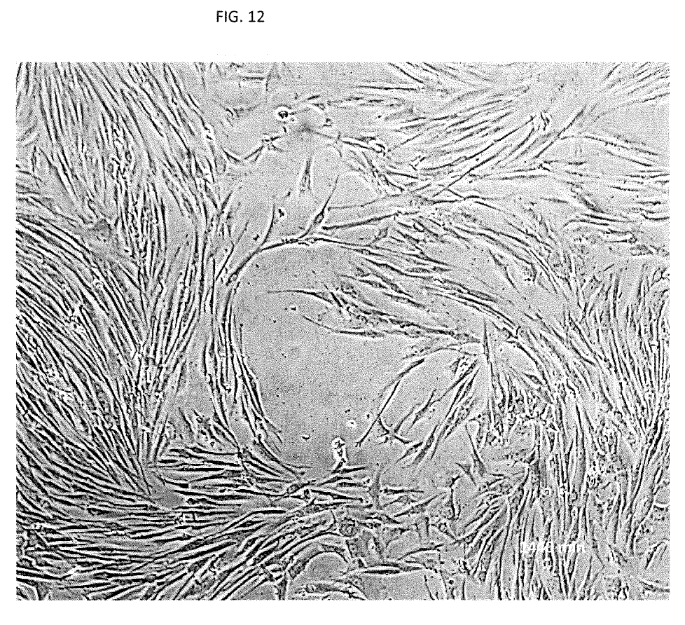

FIG. 7 (0 min), FIG. 8 (288 min), FIG. 9 (576 min), FIG. 10 (864 min), FIG. 11 (1152 min), and FIG. 12 (1440 min), show use of the GAP assay to determine ability of different culture media effect on human dermal fibroblasts to fill Gaps in confluent dells. The two dermal fibroblast cell types were cultured to confluence using the ATCC Control Media for each cell type, slow growing CCL-110 cells and rapid growing SCRC-1041 cells. The gap in the confluence was made by clearing out cells in a path made by a pipette, leaving a linear void in the cells. Then, the test media were placed on the cells after a 100% media change. A 24 hour video was recorded at 37° C. under phase microscopy taking photos every 30 to 60 seconds. The number of cells that migrated into the gap over 24 hours was counted at different times.

The figures show phase contrast photographs of GAP assay results from human dermal fibroblasts. These six phase contrast photos were taken on an EVOS® microscope (Advanced Microscopy Group, Bothell, Wash.) starting at time zero through 1440 minutes with one photo taken every minute. The six (6) time frames presented here document the degree of spreading and dividing these human dermal fibroblasts undergo in order to fill the gap in the confluence. The actual numbers of cells entering and remaining in the GAP over time are recorded below for each of the different treatments provided during the 24 hour video time frame.

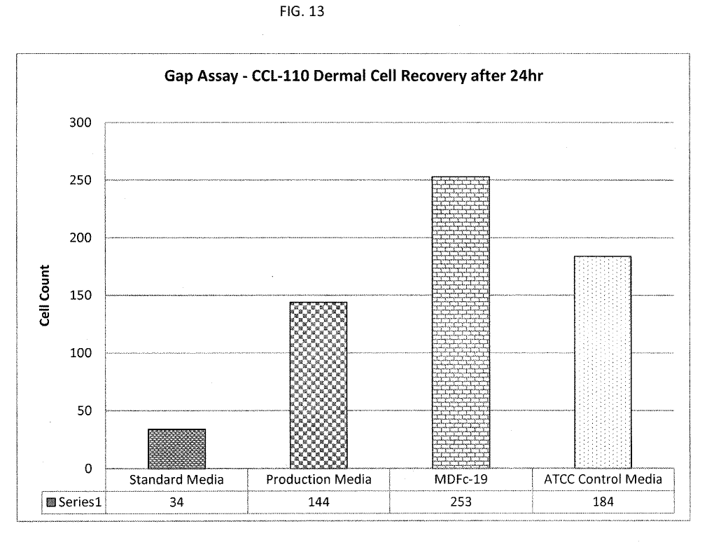

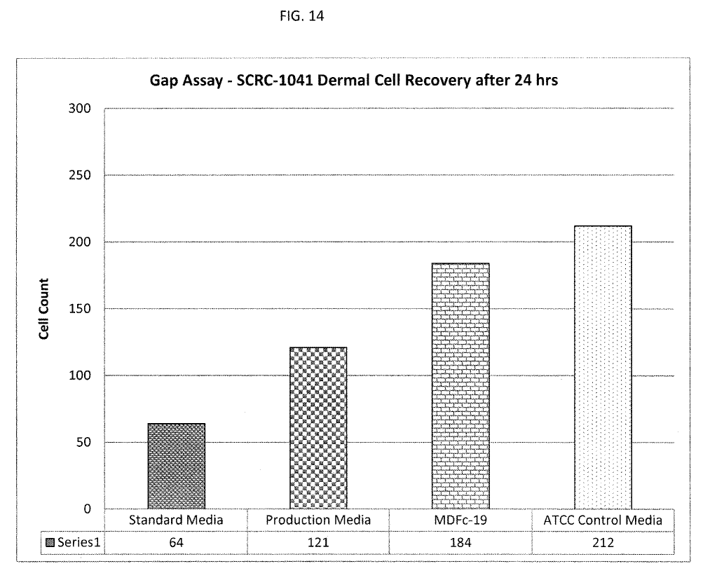

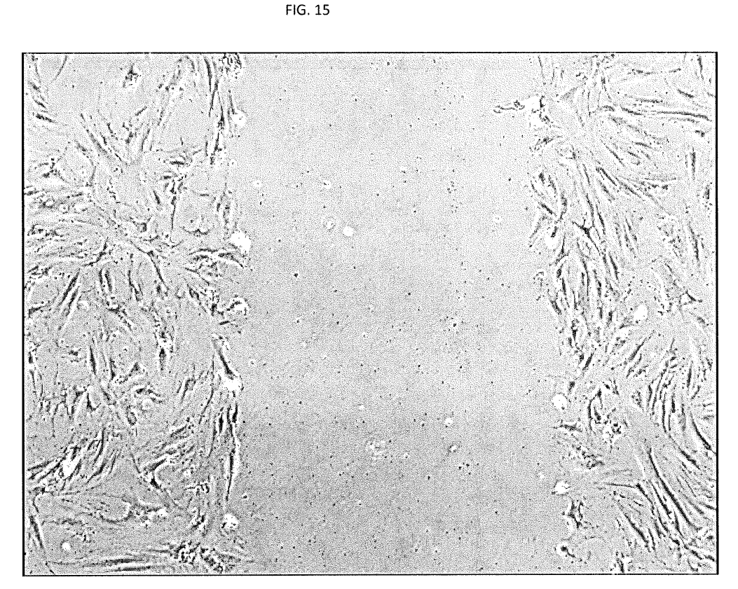

FIG. 13 (CCL-110 Cells) and FIG. 14 (SCRC-1041 Cells)

FIG. 13 (CCL-110 cells) and FIG. 14 (SCRC-1041 cells) show analysis of Gap Assay results for human dermal fibroblasts. The total number of dermal fibroblasts of each type were counted within the GAP. While there were no cells in the GAP at the start, after 24 hours for the slow growing CCL-110 cells, there were a total of 253 cells migrated into the GAP when the cells were cultured in MDFc-19 media and only 144 cells migrated when cultured in the Production Media, that represents a 76% increase in the migrating cells using the MDFc-19 media. The Standard Media culture only caused 34 cells to migrate over the same time. The ATCC control media resulted in 184 cells to migrate into the GAP. For the SCRC-1041 fast growing dermal fibroblasts, the culture with MDFc-19 brought 184 cells into the GAP compared to 121 cells for the Production Media that represents a 53% increase in the migrating cells using the MDFc-19. There were 64 migrating cells using the Standard Media. For the culture with the ATCC Control Media, 212 cells migrated into the GAP. Thus for both types of human dermal fibroblasts, the influence of MFDc-19 culture resulted in the largest numbers of fibroblasts to migrate in to fill the GAP. Again, the only difference between the Production Media and MDFc-19 media is that fact that it previously cultured growing adult human stem cells that released signals, from peptides produced with in a rapid growth rate, that are providing signals to the dermal fibroblasts to migrate and expand to fill the GAP created in the confluent cells.

FIG. 15

FIG. 15 shows phase contrast photograph of GAP results from human mesenchymal Stem Cells. Under the same 24 hours of exposure in the presence of MDFc-19, essentially no human adult stem cells migrated into the GAP. The stem cells produce and release signals that cause the dermal fibroblasts to migrate and expand to fill the GAP, but do not participate in this “repair” process by migrating themselves.

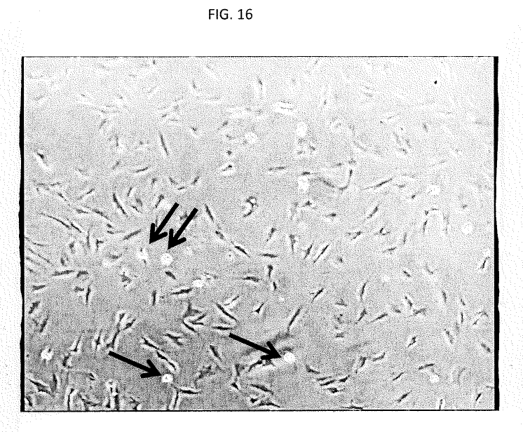

FIG. 16

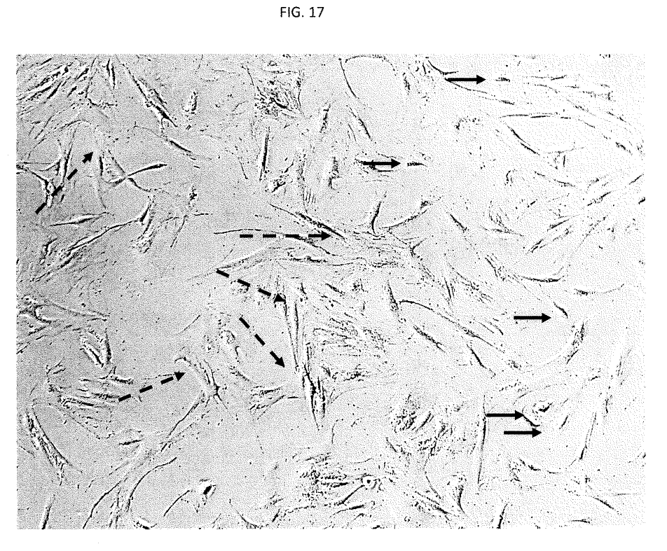

FIGS. 16 and 17 show growing human bone marrow derived stem cells cultured in production media (SCM9) supplemented with 10% FBS (fetal bovine serum). In FIG. 16, the arrows show dividing stem cells. There are four arrows. FIG. 16 shows stem cells, and is with 4× resolution under phase microscopy. Observing that when the MSC's divide, they release from the surface of the flask and round up in order to divide, split, and then settle back onto the surface to continue migrating and contacting each other. When the cells leave the surface and round up for division, the phase light turns them white. Such rounded and dividing cells are located on the photograph. The culture surface is tissue culture treated polystyrene flasks from Corning (Corning, Inc., Corning, N.Y.) with the culture media SCM9 supplemented with 10% FBS and STG. STG comprises glutathione and glutamine.

FIG. 17

In FIG. 17, the long, dashed arrows indicate Mesenchymal Stem Cells, and the short solid arrows indicate small stem cells. FIG. 17 shows stem cells with 10× resolution under phase microscopy. Images with this resolution demonstrate there are two sizes of stem cells in these preparations: Regular multi-shaped adult mesenchymal stem cells (MSC) are throughout the flask surface with multiple arms and processes as well as varying broadness that average around 6 microns (micrometers; μm) in size. The smaller stem cells that are present most frequently are bipolar with short straight processes that average around 2 microns in size. Under 30 sec video images both cells are very active with processes moving about and connecting with many other cells. The smaller stem cells are very active and connect most often with the larger stem cell processes more than with each other. The culture surface is tissue culture treated polystyrene flasks from Corning with the culture media SCM9 supplemented with 10% FBS and STG.

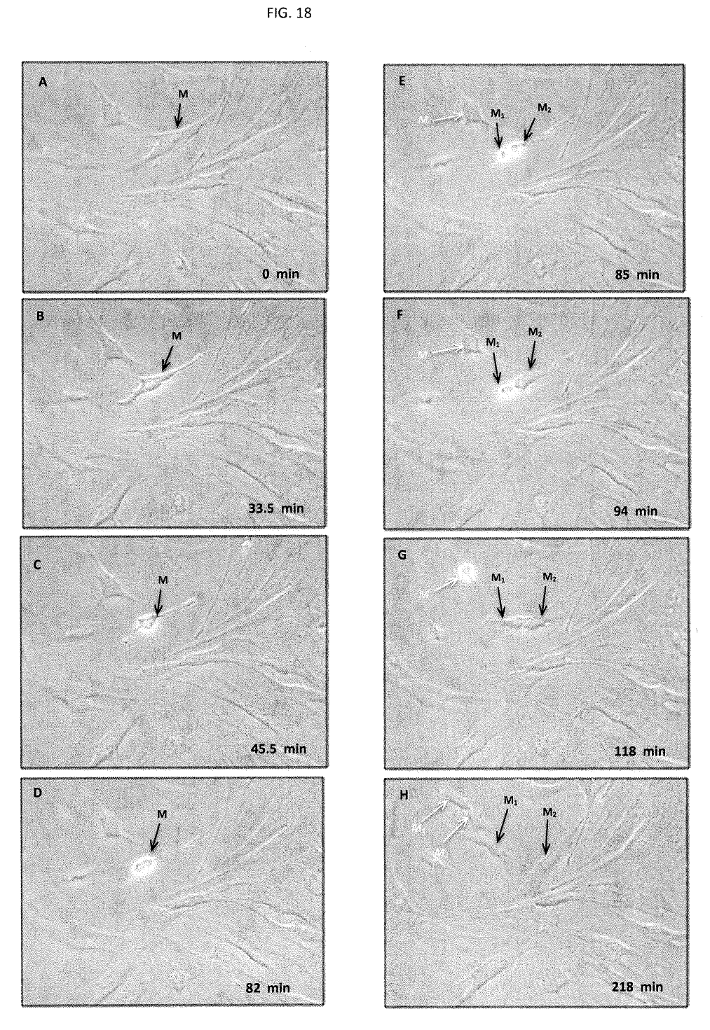

FIG. 18

FIG. 18 includes FIG. 18A (0 min), FIG. 18B (33.5 min), FIG. 18C (45.5 min), FIG. 18D (82 min), FIG. 18 E 85 min), FIG. 18F (94 min), FIG. 18G (118 min), and FIG. 18H (218 min). What is shown is stem cells and MSC Division, with 20× resolution under phase microscopy. Timed sequence of mesenchymal stem cell division by first the MSC separating from the surface of the flask and rounding up, preparing for cell division, resulting in nuclear separation, reattachment of the joined daughter cells to the flask, separation of the two cells once both reattach to the surface, and individual new cell migration away from each other. The primary MSC undergoing cell division is labeled as M with arrow in picture A and is followed throughout all the pictures until it divides into 2 cells, M₁ and M₂, starting in picture E and completed in picture H. But, during its observation, another MSC, labeled as white M starting in picture E also begins its process of cell division that is completed in the last picture as well. Its timing was not carried out to catch each of the steps as was done for the first MSC.

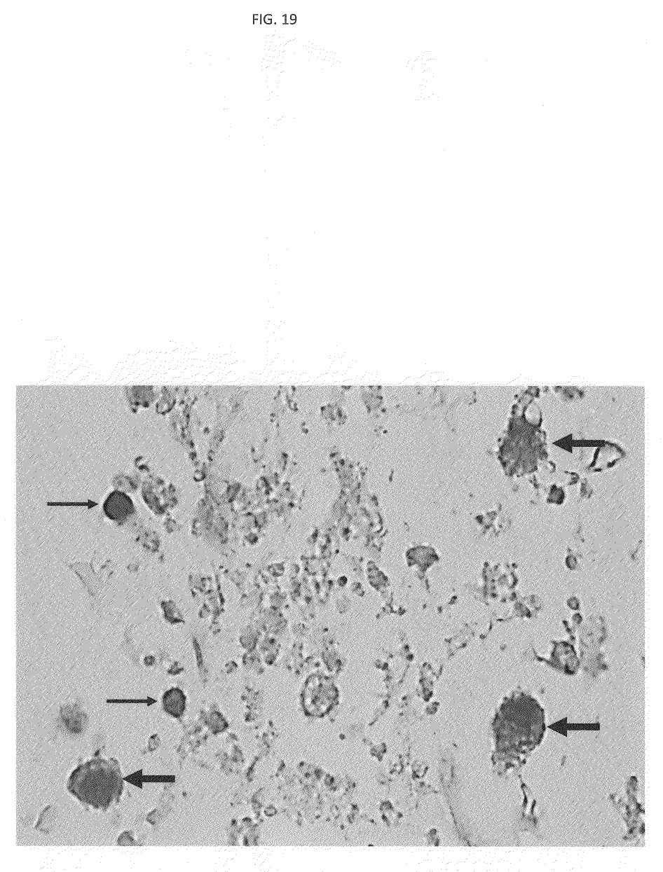

FIG. 19

FIG. 19 shows stem cells, MSCs (thick black arrows) and Small Stem Cells (thin black arrows). 100× Resolution trypsinized, fixed in formalin, paraffin blocked and sectioned with H&E staining—These cells have been treated with trypsin to remove them from the flask surface so they revert back to a round structure without processes. The larger MSC's are 6-8 microns in diameter after this treatment. The small stem cells VSEL are 2-3 microns with very little cytoplasma.

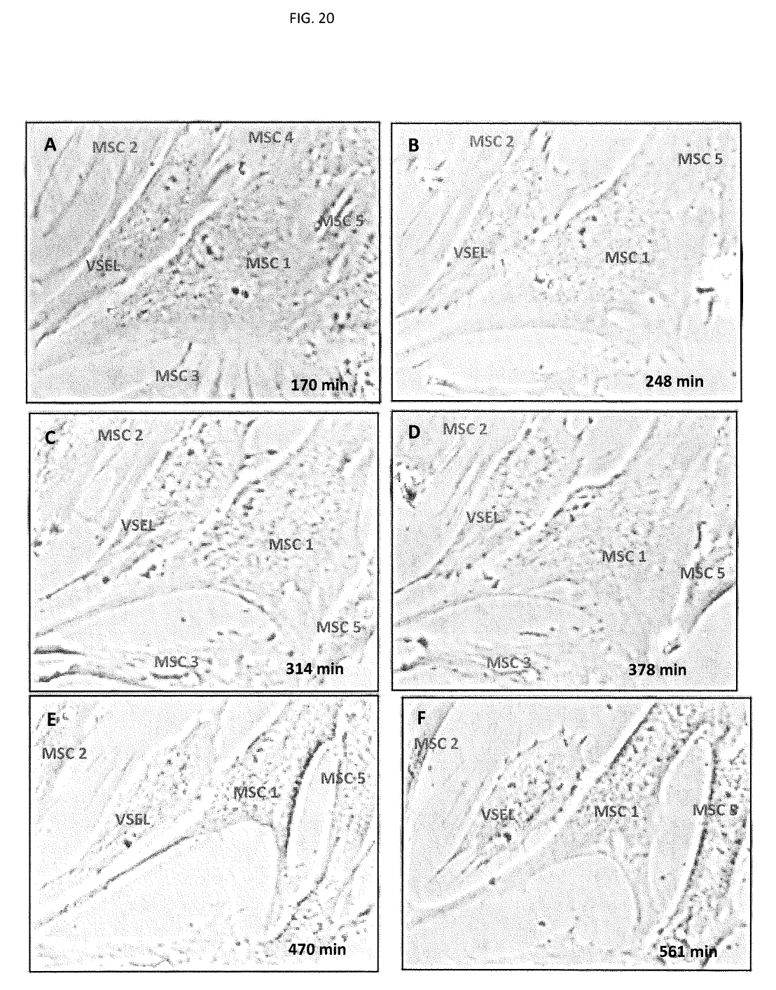

FIG. 20

FIG. 20 shows MSC contacts with other MSC's and with Small Stem Cells (VSELs), using 40× resolution under phase microscopy. The series of pictures taken over 7 hours at 37° C. with a picture taken every minute. In picture A (FIG. 20A), the small stem cell VSEL is connected to both MSC 1 and the appendage of MSC 2. VSEL is also extensively connected to MSC 4 at the top that is also connected to MSC 1. There also are a number of connections between MSC 1 with MSC 3 at the bottom of the picture and MSC 5 on the top right. In picture B (FIG. 20B), there are more dense connections between MSC 2 and the VSEL and less to MSC 2 appendage, but VSEL has withdrawn its connections to MSC 4. MSC 1 has also withdrawn most of its connections from MSC 3. There is an appendage of MSC 5 connected to MSC 1. In picture C (FIG. 20C) the connections between VSEL and MSC 1 continue with an appendage of MSC 5 connecting on the right side of MSC 1. In picture D (FIG. 20D), MSC 2 is beginning to withdraw its appendage connecting to VSEL. The appendage of MSC 5 is making a number of connections with MSC 1. In picture E (FIG. 20E), MSC 2 has completely withdrawn from the VSEL that is maintaining its connections with MSC 1 that has moved much of its volume upward off the screen. MSC 5 has broadened its appendage and reduced its connection to MSC 1 to be more at the tip but also up higher on its appendage. MSC 5 has now also connected with MSC 3. In picture F (FIG. 20F), the VSEL has changed its shape but remains tightly connected to MSC 1. MSC 1 seems to be extending a branch of its appendage to MSC 2. MSC 5 maintains its connections with MSC 1 and MSC 3. These extensive and changing MSC-MSC-VSEL-MSC connections have functional significance and are related to the production of peptides that are a key component of MDFc-19.

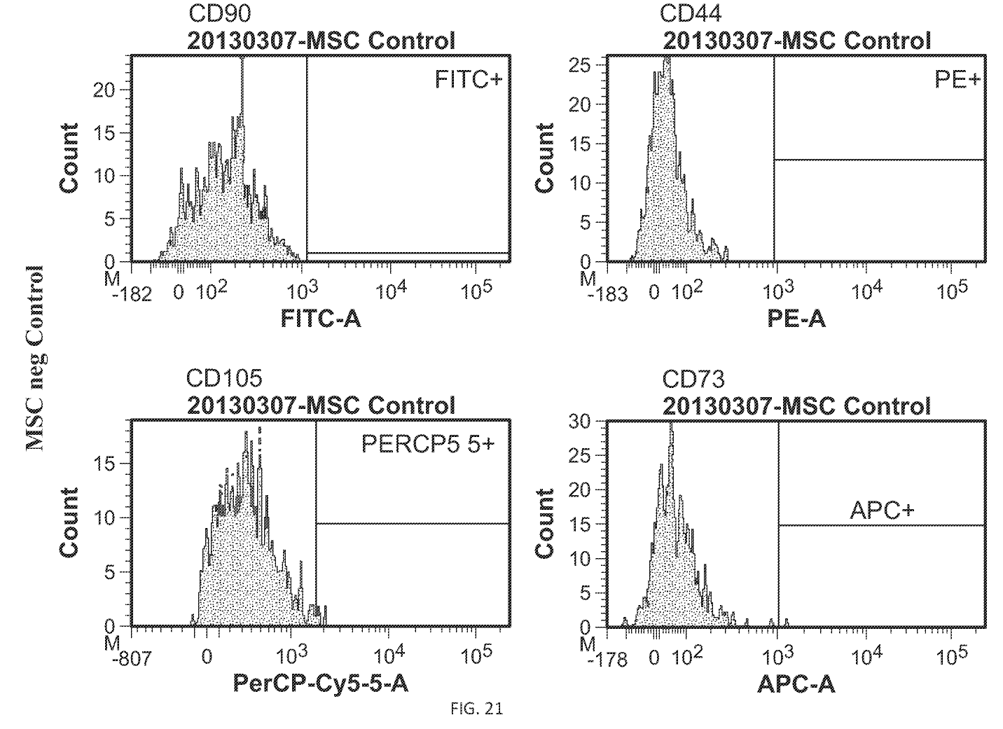

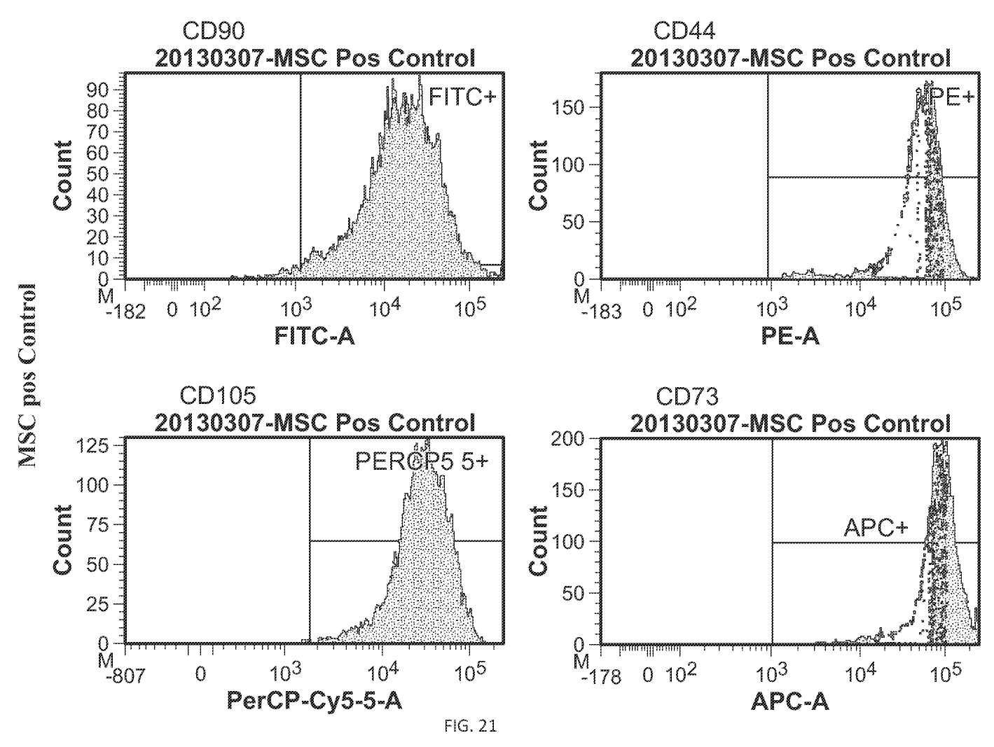

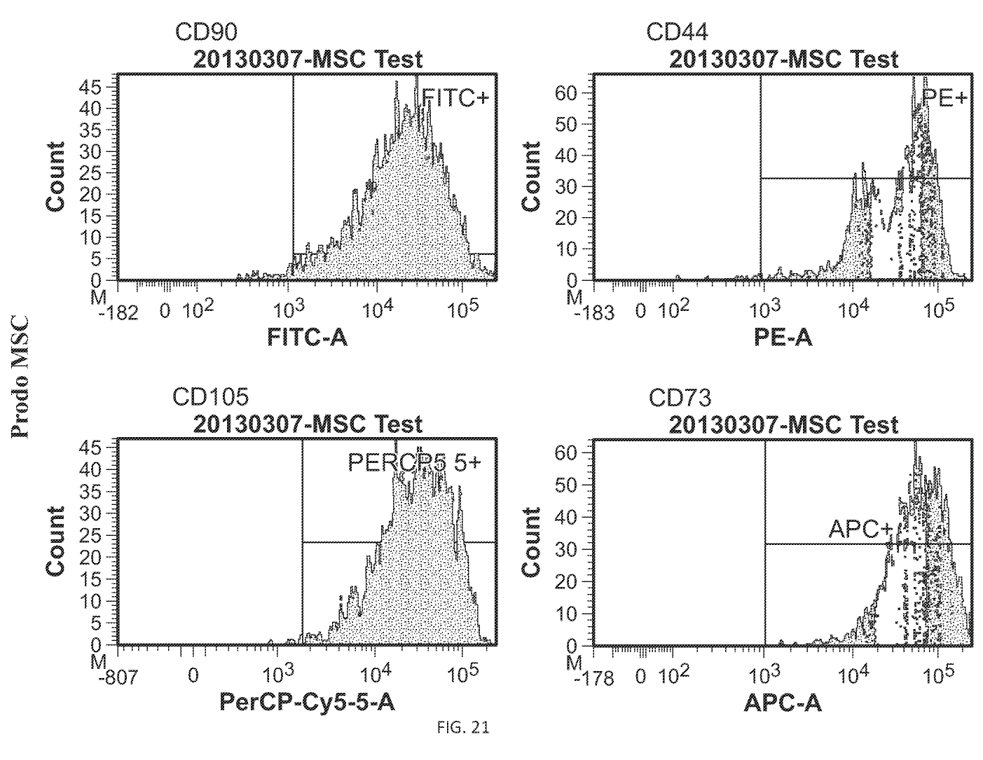

FIG. 21

FIG. 21. Flow Cytometer Testing for Morphologic Markers for MSC cells. Viable MSCs in the fresh state were used for morphologic analysis by cell markers via flow cytometer testing. The lab technician prepared the fresh cells by standard MSC protocols for testing. The assay was run with MSC negative control markers that showed that none of the MSCs of the present disclosure had markers that should not be present on human adult MSCs. The lab technician also ran MSC positive control cells for the four CD105, CD90, CD73, and CD44 cell markers that MSCs must have present on their surfaces to confirm their identity. The MSCs of the present disclosure did express these MSC positive markers on their surfaces, confirming that the appropriate human MSC's had been obtained from bone marrow, and confirming that the culture system of the present disclosure utilizing SCM9 Production Media maintains these critical markers and does not differentiate them while in cell growing culture conditions.

TABLE 1
Table 1. CMRL-1066 medium. Sigma-Aldrich (St.
Louis, MO) All quantities are in grams/liter.

L-Alanine 0.025 grams/liter
L-Arginine 0.05787
L-Aspartic Acid 0.03
L-Cysteine HCl•H₂O 0.26
L-Cystine 0.02
L-Glutamic Acid 0.075
L-Glutamine 0.1
Glycine 0.05
L-Histidine HCl•H₂O 0.02
Trans-4-Hydroxy-L-Proline 0.01
L-Isoleucine 0.02
L-Leucine 0.06
L-Lysine HCl 0.07
L-Methionine 0.015
L-Phenylalanine 0.025
L-Proline 0.04
L-Serine 0.025
L-Threonine 0.03
L-Tryptophan 0.01
L-Tyrosine 0.04
L-Valine 0.025
L-Ascorbic Acid 0.05
Paraaminobenzoic acid (PABA) 0.00005
D-Biotin 0.00001
Choline chloride 0.0005
Coenzyme A•Na 0.0025
Cocarboxylase 0.001
2′-Deoxyadenosine 0.01
2′-Deoxyguanosine 0.01
2′-Deoxycytidine•HCl 0.0116
Flavin Aadenine Dinucleotide•2Na 0.000106
Folic Acid 0.00001
myo-Inositol 0.00005
5-Methyldeoxycytidine 0.0001
beta-NAD 0.007
beta-NADP•Na 0.001
Niacinamide 0.000025
Nicotinic Acid 0.000025
D-Pantothenic Acid [hemicalcium] 0.00001
Pyridoxal•HCl 0.000025
Pyridoxine•HCl 0.000025
Riboflavin 0.00001
Thiamine•HCl 0.00001
Thymidine 0.01
Uridine-5-Triphosphate•Na 0.001
Calcium Chloride [Anhydrous] 0.2
Magnesium Sulfate [Anhydrous] 0.09769
Potassium Chloride 0.4
Sodium Acetate [Anhydrous] 0.05
Sodium Chloride 6.8
Sodium Phosphate Monobasic [Anhydrous] 0.122
D-Glucose 1.0
Phenol Red•Na 0.02124
Glutathione 0.01
D-Glucuronic Acid•Na 0.00388
Cholesterol 0.0002
Tween 80 0.005

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

### APPENDIX ONE

Liang et al (2007) In vitro scratch assay: a convenient and inexpensive method for analysis of cell migration in vitro. Nature Protocols. 2:329-333, which is hereby incorporated by reference in its entirety.

What is claimed is:

1. A composition comprising cultured cells in a medium, further comprising a first population of cultured cells in the medium that is at least 80% mesenchymal stem cells (MSCs) and a second population of cells in the medium that is at least 80% VSELs.

2. The composition of claim 1, wherein each of the MSCs are CD105+, CD90+, CD73+, CD44+.

3. The composition of claim 1, wherein each of the VSELs are SSEA-4+, Oct-4+, and Nanog+.

4. The composition of claim 1, comprising a first population of cultured cells in the medium that is at least 90% mesenchymal cells (MSCs) and a second population of cells in the medium that is at least 90% VSELs.

5. The composition of claim 1, wherein the medium is supplemented with fetal bovine serum.

6. The composition of claim 1, wherein the MSCs form filopodia or processes that contact the VSELs, wherein the VSELs form filopodia or processes that contact the MSCs, or wherein the MSCs form filopodia or processes that contact the VSELs and the VSELs form filopodia or processes that contact the MSCs.

7. The composition of claim 1, comprising a plurality of cells, wherein the plurality of cells consists of a first population of cultured cells in the medium that is at least 80% mesenchymal cells (MSCs) and a second population of cells in the medium that is at least 80% VSELs.

8. The composition of claim 1, wherein the medium is not supplemented with any purified exogenous growth factor.

9. The composition of claim 1, wherein the medium is not supplemented with purified fibroblast growth factor (FGF), or with purified hepatocyte growth factor (HGF), or with any analogue thereof.

10. A fluid reagent prepared by a method comprising the step of incubating together for a predetermined period of time, in a medium, a first population of cultured cells that is at least 80% mesenchymal cells (MSCs) and a second population of cells in the medium that is at least 80% VSELs, followed the step of storing the fluid reagent as it occurs following the incubating for the predetermined period of time.

11. The fluid reagent of claim 10 that is cell free.

12. The fluid reagent of claim 10 that is not cell free.

13. The fluid reagent of claim 10, that is capable of stimulating confluence of human dermal fibroblasts, capable of stimulating closing of a gap in an assay of human dermal fibroblasts, and capable of stimulating expression of collagen expression by the human dermal fibroblasts.

14. The fluid reagent of claim 10, that is:
(a) Capable of stimulating confluence of human dermal fibroblasts;
(b) capable of stimulating closing of a gap in an assay of human dermal fibroblasts as determined by GAP assay method of Example 1; and
(c) Capable of stimulating expression of pro-collagen type-1 expression by the human dermal fibroblasts.

15. The fluid reagent of claim 10, that is:
(a) Capable of stimulating confluence of human dermal fibroblasts;
(b) Capable of stimulating closing of a gap in an assay of human dermal fibroblasts as determined by GAP assay method of Example 1 wherein the gap at time=zero hours is at least 100 micrometers (μm) wide and wherein the number of cells in the gap at time=greater than 24 hours is greater than 150 cells; and
(c) Capable of stimulating expression of pro-collagen type-1 expression by the human dermal fibroblasts.

16. The fluid reagent of claim 10, that is:
(a) Capable of stimulating confluence of human dermal fibroblasts;
(b) Capable of stimulating closing of a gap in an assay of human dermal fibroblasts as determined by GAP assay method of Example 1 wherein the gap at time=zero hours is at about 100 micrometers (μm) wide and wherein the number of cells in the gap at time=greater than 24 hours is greater than 200 cells; and
(c) Capable of stimulating expression of pro-collagen type-1 expression by the human dermal fibroblasts.

17. The fluid reagent of claim 10 in combination with a pharmaceutically acceptable excipient.

18. The fluid reagent of claim 10, further comprising separating the fluid reagent into a first fraction that comprises one or more peptides that are:
(a) Capable of stimulating confluence of human dermal fibroblasts;
(b) Capable of stimulating closing of a gap in an assay of human dermal fibroblasts as determined by GAP assay method of Example 1 wherein the gap at time=zero hours is at about 100 micrometers (μm) wide and wherein the number of cells in the gap at time=greater than 24 hours is greater than 200 cells; and
(c) Capable of stimulating expression of pro-collagen type-1 expression by the human dermal fibroblasts;
a second fraction that does not contain said one or more peptides, and discarding the second fraction.

19. A method for stimulating growth of at least one human dermal fibroblast, comprising contacting a growth stimulatory amount of the fluid reagent of claim 10 to:
(a) The skin of a subject, wherein the contacting is topical; or (b) To a wound of a subject.

20. The method of claim 19, wherein the subject is a human subject.

21. The method of claim 18, wherein the skin comprises one or more of age-induced wrinkles, ultraviolet light-induced wrinkles, subdermal ultraviolet light-induced damage, and scar tissue.

22. The method of claim 18, that results in one or more of reduction of age-induced wrinkles, reduction of ultraviolet light-induced wrinkles, reduction of subdermal ultraviolet light-induced damage, reduction in scar tissue, and increase in dermal thickness.

22. The method of claim 19, wherein the fluid reagent of claim 10 further comprises an excipient that facilitates absorption of peptides by the skin.

23. The method of claim 19, wherein the subject is mammalian.

24. The method of claim 19, wherein the subject is human, a veterinary subject, or an agricultural livestock subject.

25. A composition comprising cultured cells in a medium, further comprising a first population of cultured cells in the medium that is at least 80% mature mesenchymal stem cells (MSCs) and a second population of cells in the medium that is at least 80% small cells, wherein the small cells are recently divided MSCs or progenitors of MSCs.

26. The composition of claim 25, wherein each of the mature MSCs is CD105+, CD90+, CD73+, CD44+.

27. A fluid reagent prepared by a method comprising the step of:
incubating together for a predetermined period of time, in a medium, a first population of cultured cells that is at least 80% mesenchymal stem cells (MSCs) and a second population of cells in the medium that is at least 80% small cells that are recently divided MSCs or progenitors of MSCs, followed the step of:
storing the fluid reagent following the incubating for the predetermined period of time.

28. The fluid reagent of claim 27, wherein the storing is in a cool environment, wherein the storing is stored frozen, or wherein the storing is stored in a dried or dessicated state.
